(19) United States
(12) Patent Application Publication
(10) Pub. No.: US 2009/0305369 A1
(43) Pub. Date: Dec. 10, 2009

(54) DELETION MUTANTS FOR THE PRODUCTION OF ISOBUTANOL

(75) Inventors: Gail K. Donaldson, Newark, DE (US); Lori Ann Maggio-Hall, Wilmington, DE (US); Charles E. Nakamura, Claymont, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I.DU PONT DE NEMOURS AND COMPANY, Wilmington, DE (US)

(21) Appl. No.: 12/477,946

(22) Filed: Jun. 4, 2009

Related U.S. Application Data
(60) Provisional application No. 61/058,568, filed on Jun. 4, 2008.

(51) Int. Cl. C12P 7/16; C12N 1/21

(52) U.S. Cl. 435/160; 435/252.3; 435/252.33

(57) ABSTRACT

An E. coli host strain was engineered wherein genes adhE, IdhA, frdB, and pfIB were disrupted and novel butanol dehydrogenase gene, sadB, from Achromobacter xylosoxidans, was added to produce the isobutanol production host.

This application claims the benefit of U.S. Provisional Application No. 61/058568, filed Jun. 4, 2008, the disclosure of which is hereby incorporated in its entirety.

### FIELD OF THE INVENTION

The invention relates to the field of microbiology and molecular biology. More specifically the invention describes an enteric deletion mutant having an enhanced ability to produce isobutanol.

### BACKGROUND OF THE INVENTION

Butanol is an important industrial chemical, useful as a fuel additive, as a feedstock chemical in the plastics industry, and as a food grade extractant in the food and flavor industry. Each year 10 to 12 billion pounds of butanol are produced by petrochemical means and the need for this commodity chemical will likely increase. While the known chemical synthesis of isobutanol via petroleum feedstocks are expensive and are not environmentally friendly, production of isobutanol from plant-derived raw materials would minimize green house gas emissions and would represent an advance in the art.

Isobutanol is produced biologically as a by-product of incomplete metabolism of amino acids, specifically L-valine, during yeast fermentation. Following metabolism of the amine group of L-valine as a nitrogen source, the resulting α-keto acid is decarboxylated and reduced to isobutanol, albeit at very low yields, via the Ehrlich pathway. For example, the concentration of isobutanol produced in beer fermentation is less than 16 parts per million.

The economical biosynthesis of isobutanol directly from sugars would represent an environmentally responsible, cost-effective process for the production of isobutanol as a single product. In a copending and commonly owned US application (US20070092957) a strain overexpressing all the necessary enzyme activities for conversion of glucose to isobutanol was disclosed and isobutanol production in low concentrations (0.3˜10 mM) was demonstrated.

Recently Atsumi, S., et al., (Nature 451:86-90, 2008) described development of a recombinant E. coli strain which produced isobutanol in concentrations up to 300 mM. This recombinant E.coli was disrupted in genes adhE, IdhA, frdBC, fnr, pta and pfIB and two plasmids bearing an isobutanol biosynthetic pathway similar to that described in the commonly owned and co-pending US Application 20070092957. These plasmids carried an acetolactate synthase, an acetohydroxy acid reductoisomerase, an acetohydroxy acid dehydratase, a 2-keto acid decarboxylase and an alcohol dehydrogenase. A reading of Atsumi et al. (supra) implies that host cells having isobutanol biosynthetic pathways may obtain enhanced isobutanol production where genes, key to competing carbon pathways are disrupted. However, is appears that the host cell of Atsumi et al. (supra) has a far greater number of genetic modifications than is needed to achieve enhanced isobutanol production. The greater the number of genetic modifications in fundamental endogenous carbon pathways increases the likelihood of poor host cell metabolism, which will ultimately compromise the cells' use as a production host.

There is a need therefore for a host cell having a minimum number of genetic modifications in its endogenous carbon pathways for the production of isobutanol. Applicants have solved the stated problem, describing here an enteric bacterial host cell having disruptions in only 4 genes in endogenous carbon pathways, resulting in an enhanced yield of isobutanol.

### SUMMARY OF THE INVENTION

The present invention describes an enteric bacterial production host for the production of isobutanol. The host cell preferably contains an isobutanol biosynthetic pathway that utilizes a butanol dehydrogenase (secondary alcohol dehydrogenase, sadB) in the final step of the production of butanol and contains genetic modifications in endogenous carbon pathways that leaves the cell free of at least one of the following enzyme activities: 1) pyruvate formate lyase (EC 2.3.1.54), 2) fumarate reductase enzyme complex (EC 1.3.99.1), 3) Alcohol dehydrogenase (EC 1.2.1.10-acetaldehyde dehydrogenase and EC 1.1.1.1-alcohol dehydrogenase), and 4) lactate dehydrogenase (EC 1.1.1.28). Enteric hosts having disruptions in these enzyme activities demonstrate improved rates of isobutanol as compared with similar hosts not having these disruptions.

Accordingly the invention provides an enteric production host for the production of isobutanol comprising at least one gene encoding a polypeptide having butanol dehydrogenase activity wherein the host produces isobutanol and is substantially free of at least one of the following enzyme activities:

a) Pyruvate formate lyase (EC 2.3.1.54)

b) Fumarate reductase enzyme complex (EC 1.3.99.1),

c) Alcohol dehydrogenase (EC 1.2.1.10/EC 1.1.1.1.)

d) Lactate dehydrogenase (EC 1.1.1.28)

In another embodiment the invention provides that the host cell of the invention comprise at least one gene encoding a polypeptide having butanol dehydrogenase activity where the polypeptide has at least 90% identity to the amino acid sequence as set forth in SEQ ID NO: 10 over a length of 348 amino acids using BLAST with scoring matrix BLOSUM62, an expect cutoff of 10 and word size 3 and a gap opening penalty of 11 and a gap extension of 1.

In another embodiment the invention provides a host cell comprising an isobutanol biosynthetic pathway comprising:
a) at least one gene encoding an acetolactate synthase having the EC number 2.2.1.6 9 for the conversion of pyruvate to acetolactate:
b) at least one gene encoding acetohydroxy acid isomeroreductase EC number 1.1.1.86 for the conversion of acetolactate to 2,3-dihydroxyisovalerate;
c) at least one gene encoding acetohydroxy acid dehydratase EC number 4.2.1.9 for the conversion of 2,3-dihydroxyisovalerate to α-ketoisovalerate;
d) at least one gene encoding a branched-chain keto acid decarboxylase EC number 4.1.1.72 for the conversion of α-ketoisovalerate to isobutyraldehyde; and
e) at least one gene encoding a butanol dehydrogenase polypeptide where that gene is isolated from A. xylosoxidans.

In another embodiment the invention comprises a method for the production of isobutanol comprising growing the production host of the invention in a fermentation medium comprising a carbon substrate under conditions wherein isobutanol is produced.

### BRIEF DESCRIPTION OF THE FIGURE AND SEQUENCE DESCRIPTIONS

The invention can be more fully understood from the following detailed description, figure, and the accompanying sequence descriptions, which form a part of this application.

FIGS. 1A and 1B depict the isobutanol biosynthetic pathway of this invention comprised of steps labeled “a”, “b”, “c”, “d”, and “e” and represent the substrate to product conversion described below. Reactions “f” through “i” represent the specific four reactions that have been disrupted in this disclosure to prevent consumption of pyruvate for side reactions that reduce its availability for isobutanol synthesis.

The following sequences conform with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

Nucleotide and amino acid sequences of the invention are listed in Tables 1 and 2 below:

TABLE 1
Summary of gene and protein SEQ ID numbers

| | SEQ ID NO: | SEQ ID NO: |
|---|---|---|
| Description | Nucleic acid | Peptide |
| Klebsiella pneumoniae | 1 | 2 |
| budB; (acetolactate | | |
| synthase) | | |
| Escherichia coli | 3 | 4 |
| ilvC; (acetohydroxy acid | | |
| reductoisomerase) | | |
| Escherichia coli | 5 | 6 |
| ilvD; (acetohydroxy acid | | |
| dehydratase) | | |
| Lactococcus lactis | 7 | 8 |
| kivD; (branched-chain | | |
| α-keto acid | | |
| decarboxylase), codon | | |
| optimized | | |
| Achromobacter | 9 | 10 |
| xylosoxidans; sadB; | | |
| butanol dehydrogenase | | |
| Bacillus subtilis | 12 | 11 |
| (acetolactate synthase) | | |
| Lactococcus lactis | 14 | 13 |
| (acetolactate synthase) | | |
| Saccharomyces | 16 | 15 |
| cerevisiae | | |
| (acetohydroxy acid | | |
| isomeroreductase) | | |
| Methanococcus | 18 | 17 |
| maripaludis | | |
| (acetohydroxy acid | | |
| isomeroreductase) | | |
| Bacillus subtilis | 20 | 19 |
| (acetohydroxy acid | | |
| isomeroreductase) | | |
| Saccharomyces | 22 | 21 |
| cerevisiae | | |
| (acetohydroxy acid | | |
| dehydratase) | | |
| Methanococcus | 24 | 23 |
| maripaludis | | |
| (acetohydroxy acid | | |
| dehydratase) | | |
| Bacillus subtilis | 26 | 25 |
| (acetohydroxy acid | | |
| dehydratase) | | |
| Lactococcus lactis AAS | 28 | 27 |
| (branched-chain keto | | |
| acid decarboxylase) | | |
| Lactococcus lactis AJ | 29 | 8 |
| (branched-chain keto | | |
| acid decarboxylase) | | |
| Salmonella typhimurium | 31 | 30 |
| (indole pyruvate | | |
| decarboxylase) | | |
| Clostridium | 33 | 32 |
| acetobutylicum | | |
| (pyruvate | | |
| decarboxylase) | | |
| Escherichia coli K-12 | 47 | 46 |
| MG1655; pflB | | |
| (pyruvate formate lyase) | | |
| Escherichia coli K-12 | 49 | 48 |
| MG1655; frdB | | |
| (fumarate reductase) | | |
| Escherichia coli K-12 | 55 | 54 |
| MG1655; frdA | | |
| (fumarate reductase) | | |
| Escherichia coli K-12 | 57 | 56 |
| MG1655; frdC | | |
| (fumarate reductase) | | |
| Escherichia coli K-12 | 59 | 58 |
| MG1655; frdD | | |
| (fumarate reductase) | | |
| Escherichia coli K-12 | 53 | 52 |
| MG1655 | | |
| adhE (alcohol | | |
| dehydrogenase) | | |
| Escherichia coli K-12 | 51 | 50 |
| MG1655; IdhA | | |
| (lactate dehydrogenase) | | |

TABLE 2
Primers used in the application

| | | Gene- | SEQ |
|---|---|---|---|
| Name | Sequence | specific | ID NO: |
| pfIB CkUp | TCATCACTGATAACCTGATTCCGG | pfIB | 34 |
| pfIB CkDn | CGAGTCTGTTTTGGCAGTCACCTTAA | pfIB | 35 |
| frdB CkUp | GAGCGTGACGACGTCAACTTCCT | frdB | 36 |
| frdB CkDn | CAGTTCAATGCTGAACCACACAG | frdB | 37 |
| IdhA CkUp | GAAGGTTGCGCCTACACTAAGCA | IdhA | 38 |
| IdhA CkDn | GGGAGCGGCAAGATTAAACCAGT | IdhA | 39 |
| adhE CkUp | TGGATCACGTAATCAGTACCCAG | adhE | 40 |
| adhE CkDn | ATCCTTAACTGATCGGCATTGCC | adhE | 41 |
| N695A | GACCTAGGAGGTCACACATGAAAGCT | sadB | 42 |
| | CTGG | forward | |
| | | w/ AvrII | |
| | | and RBS | |
| N696A | CGACTCTAGAGGATCCCCGGGTACC | sadB | 43 |
| | | reverse | |
| | | w/ XbaI | |
| | | site | |
| N473 | GGAATTCACA CATGAAAGCT | Forward | 44 |
| | CTGGTTTATC | primer | |
| N469 | GCGTCCAGGG CGTCAAAGAT | Reverse | 45 |
| | CAGGCAGC primer | | |

### DETAILED DESCRIPTION OF THE INVENTION

The present disclosure describes development of a novel production host combining a set of pathway elements and deletions to produce unexpectedly high levels of isobutanol (e.g. 35 g/L) under extractive fermentation conditions. This disclosure describes an E. coli strain which was disrupted in genes adhE, IdhA, frdB, and pfIB. The pTrc99A::budB-ilvC-ilvD-kivD plasmid described in the commonly owned US Application 20070092957 was modified with addition of a butanol dehydrogenase from Achromobacter xylosoxidans, sadB, to produce the isobutanol production host. The present disclosure meets a number of commercial and industrial needs. Butanol is an important industrial commodity chemical with a variety of applications, where its potential as a fuel or fuel additive is particularly significant. Although only a four-carbon alcohol, butanol has an energy content similar to that of gasoline and can be blended with any fossil fuel. Butanol is favored as a fuel or fuel additive as it yields only C0₂ and little or no SO_{X} or NO_{X} when burned in the standard internal combustion engine. Additionally butanol is less corrosive Additionally, the present disclosure describes the production of isobutanol from plant derived carbon sources, avoiding the negative environmental impact associated with standard petrochemical processes for butanol production.

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “invention” or “present invention” as used herein is a non-limiting term and is not intended to refer to any single embodiment of the particular invention but encompasses all possible embodiments as described in the specification and the claims.

The term “isobutanol biosynthetic pathway” refers to an enzymatic pathway to produce isobutanol. Exemplary isobutanol biosynthetic pathways are discussed and described in commonly owned and co-pending US Application 20070092957A1, incorporated herein by reference in its entirety.

The term “knockout” refers to disruption of a particular gene in a plasmid or a microorganism to render that particular gene dysfunctional. In the present disclosure, genes adhE, IdhA, frdB, and pfIB were knocked out in the host strain for isobutanol production.

The term “pfIB” refers to the gene encoding the pyruvate formate lyase enzyme which converts pyruvate to formate.

The term “frdABCD: refers to an operon which encodes the fumarate reductase enzyme complex which converts succinate to fumarate.

The term “IdhA” refers to the gene encoding the lactate dehydrogenase enzyme and converts pyruvate to pactate.

The term “adhE” refers to the gene encoding the pyruvate formate lyase enzyme which converts acetyl-CoA to ethanol.

The terms “acetolactate synthase” and “acetolactate synthetase” are used intechangeably herein to refer to an enzyme that catalyzes the conversion of pyruvate to acetolactate and CO₂. Preferred acetolactate synthases are known by the EC number 2.2.1.6 9 (Enzyme Nomenclature 1992, Academic Press, San Diego). These enzymes are available from a number of sources, including, but not limited to, Bacillus subtilis (GenBank Nos: CAB15618 (SEQ ID NO:11), Z99122 (SEQ ID NO:12), NCBI (National Center for Biotechnology Information) amino acid sequence, NCBI nucleotide sequence, respectively), Klebsiella pneumoniae (GenBank Nos: AAA25079 (SEQ ID NO:2), M73842 (SEQ ID NO:1), and Lactococcus lactis (GenBank Nos: AAA25161 (SEQ ID NO:13), L16975 (SEQ ID NO:14).

The terms “acetohydroxy acid isomeroreductase” and “acetohydroxy acid reductoisomerase” are used interchangeably herein to refer to an enzyme that catalyzes the conversion of acetolactate to 2,3-dihydroxyisovalerate using NADPH (reduced nicotinamide adenine dinucleotide phosphate) as an electron donor. Preferred acetohydroxy acid isomeroreductases are known by the EC number 1.1.1.86 and sequences are available from a vast array of microorganisms, including, but not limited to, Escherichia coli (GenBank Nos: NP_{—}418222 (SEQ ID NO:4), NC_{—}000913 (SEQ ID NO:3)), Saccharomyces cerevisiae (GenBank Nos: NP_{—}013459 (SEQ ID NO:15), NC_{—}001144 (SEQ ID NO:16), Methanococcus maripaludis (GenBank Nos: CAF30210 (SEQ ID NO:17), BX957220 (SEQ ID NO:18), and Bacillus subtilis (GenBank Nos: CAB14789 (SEQ ID NO:19), Z99118 (SEQ ID NO:20).

The term “acetohydroxy acid dehydratase” refers to an enzyme that catalyzes the conversion of 2,3-dihydroxyisovalerate to α-ketoisovalerate. Preferred acetohydroxy acid dehydratases are known by the EC number 4.2.1.9. These enzymes are available from a vast array of microorganisms, including, but not limited to, E. coli (GenBank Nos: YP_{—}026248 (SEQ ID NO:6), NC_{—}000913 (SEQ ID NO:5), S. cerevisiae (GenBank Nos: NP_{—}012550 (SEQ ID NO:21), NC_{—}001142 (SEQ ID NO:22), M. maripaludis (GenBank Nos: CAF29874 (SEQ ID NO:23), BX957219 (SEQ ID NO:24), and B. subtilis (GenBank Nos: CAB14105 (SEQ ID NO:25), Z99115 (SEQ ID NO:26).

The term “branched-chain α-keto acid decarboxylase” refers to an enzyme that catalyzes the conversion of α-ketoisovalerate to isobutyraldehyde and CO₂. Preferred branched-chain α-keto acid decarboxylases are known by the EC number 4.1.1.72 and are available from a number of sources, including, but not limited to, Lactococcus lactis (GenBank Nos: AAS49166 (SEQ ID NO:27), AY548760 (SEQ ID NO:28); CAG34226 (SEQ ID NO:8), AJ746364 (SEQ ID NO:29), Salmonella typhimurium, which is also known as indolepyruvate decarboxylase, (GenBank Nos: NP_{—}461346 (SEQ ID NO:30), NC_{—}003197 (SEQ ID NO:31), and Clostridium acetobutylicum, which is also known as pyruvate decarboxylase, (GenBank Nos: NP_{—}149189 (SEQ ID NO:32), NC_{—}001988 (SEQ ID NO:33).

The terms “butanol dehydrogenase” and “secondary alcohol dehydrogenase”, are used interchangeably here, and refer to the enzymes that occur in many microorganisms, facilitate the interconversion between alcohols and aldehydes or ketones with the reduction of NAD⁺ to NADH. The preferred example of such an enzyme is the butanol dehydrogenase from Achromobacter xylosoxidans (nucleotide SEQ ID NO: 9 and amino acid SEQ ID NO: 10). The A. xylosoxidans sadB enzyme catalyzes the conversion of isobutyraldehyde to isobutanol.

The term “carbon substrate” or “fermentable carbon substrate” refers to a carbon source capable of being metabolized by host microorganisms of the present invention and particularly carbon sources selected from the group consisting of monosaccharides, such as glucose or fructose; disaccharides, such as lactose or sucrose; oligosaccharides; polysaccharides, such as starch or cellulose; one carbon substrates; and mixtures thereof.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of a microorganism. A “foreign gene” or “heterologous gene” refers to a gene not normally found in the host microorganism, but that is introduced into the host microorganism by gene transfer. Foreign genes can comprise native genes inserted into a non-native microorganism, or chimeric genes. A “transgene” is a gene that has been introduced into the genome by a transformation procedure.

As used herein, an “isolated nucleic acid fragment” or “isolated nucleic acid molecule” or “genetic construct” will be used interchangeably and will mean a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

As used herein the term “coding sequence” refers to a DNA sequence that encodes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

As used herein the term “transformation” refers to the transfer of a nucleic acid fragment into a host microorganism, resulting in genetically stable inheritance. Host microorganisms containing the transformed nucleic acid fragments are referred to as “transgenic” or “recombinant” or “transformed” microorganisms.

The term “plasmid” refers to an extra chromosomal element often carrying genes that are not part of the central metabolism of the cell, and usually in the form of circular double-stranded DNA molecules. Such elements may be autonomously replicating sequences, genome integrating sequences, phage or nucleotide sequences, linear or circular, of a single or double stranded DNA or RNA, derived from any source, in which a number of nucleotide sequences have been joined or recombined into a unique construction which is capable of introducing an expression cassette(s) into a cell, wherein said expression cassette(s) comprise the coding sequence of a selected gene and regulatory sequences preceding (5′ non-coding sequences) and following (3′ non-coding sequences) the coding sequence that are required for expression of the selected gene product.

The term “codon-optimized” as it refers to genes or coding regions of nucleic acid molecules for transformation of various hosts, refers to the alteration of codons in the gene or coding regions of the nucleic acid molecules to reflect the typical codon usage of the host microorganism without altering the polypeptide encoded by the DNA.

As used herein, the terms “transduction” and “generalized transduction” are used interchangeably and refer to a phenomenon in which bacterial DNA is transferred from one bacterial cell (the donor) to another (the recipient) by a phage particle containing bacterial DNA.

The terms “P1 donor cell” and “donor cell” are used interchangeably and refer to a bacterial strain susceptible to infection by a bacteriophage or virus, and which serves as a source for the nucleic acid fragments packaged into the transducing particles. Typically, the genetic make up of the donor cell is similar or identical to the “recipient cell” which serves to receive P1 lysate containing transducing phage or virus produced by the donor cell.

The terms “P1 recipient cell” and “recipient cell” are used interchangeably and refer to a bacterial strain susceptible to infection by a bacteriophage or virus and which serves to receive lysate containing transducing phage or virus produced by the donor cell.

The term “chaotropic agent”, means a substance which disrupts the three dimensional structure in macromolecules such as proteins, DNA, or RNA.

The term “azeotropic” refers to a mixture of two or more pure chemicals in such a ratio that its composition cannot be changed by simple distillation.

The term “pervaporation” refers to a method for the separation of mixtures of liquids by partial vaporization through a non-porous or porous membrane.

The term “hydrophilic” refers to a physical property of a molecule that can transiently bond with water (H₂O) through hydrogen bonding.

The term “substantially free” when used in reference to the presence or absence of enzyme activities (e.g., pyruvate formate lyase, fumarate reductase, alcohol dehydrogenase and lactate dehydrogenase) in carbon pathways that compete with the present isobutanol pathway means that the level of the enzyme is substantially less than that of the same enzyme in the wildtype host, where less than 50% of the wildtype level is preferred and less than about 90% of the wildtype level is most preferred.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1) Computational Molecular Biology (Lesk, A. M., Ed.) Oxford University, Press, NY (1988); 2) Biocomputing: Informatics and Genome Projects (Smith, D. W., Ed.) Academic Press, NY (1993); 3) Computer Analysis of Sequence Data, Part I (Griffin, A. M., and Griffin, H. G., Eds.) Humania Press, NJ (1994); 4) Sequence Analysis in Molecular Biology (von Heinje, G., Ed.) Academic Press (1987); and 5) Sequence Analysis Primer (Gribskov, M. and Devereux, J., Eds.) Stockton, N.Y. (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V described by Higgins and Sharp, (CABIOS. 5:151-153, 1989); and Higgins, D. G. et al., (Comput. Appl. Biosci., 8:189-191, 1992) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W described by Higgins and Sharp, (supra); Higgins, D. G. et al., (supra) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 24%, 30%, 35%, 40%, 45%, 50%, 55%, 60%, 65%, 70%, 75%, 80%, 85%, 90%, or 95%, or any integer percentage from 24% to 100% may be useful in describing the present invention, such as 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1) the GCG suite of programs (Wisconsin Package Version 9.0, Genetics Computer Group (GCG), Madison, Wis.); 2) BLASTP, BLASTN, BLASTX (Altschul et al., J. Mol. Biol., 215:403-410, 1990); 3) DNASTAR (DNASTAR, Inc. Madison, Wis.); 4) Sequencher (Gene Codes Corporation, Ann Arbor, Mich.); and 5) the FASTA program incorporating the Smith-Waterman algorithm (W. R. Pearson, Comput. Meth. Gen. Res., [Proc. lnt. Symp.], Meeting Date 1992, 111-120, 1994. Editor(s): Suhai, Sandor. Plenum Press, New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related microorganisms), to highly similar fragments (such as genes that duplicate functional enzymes from closely related microorganisms).

Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., supra). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular fungal proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

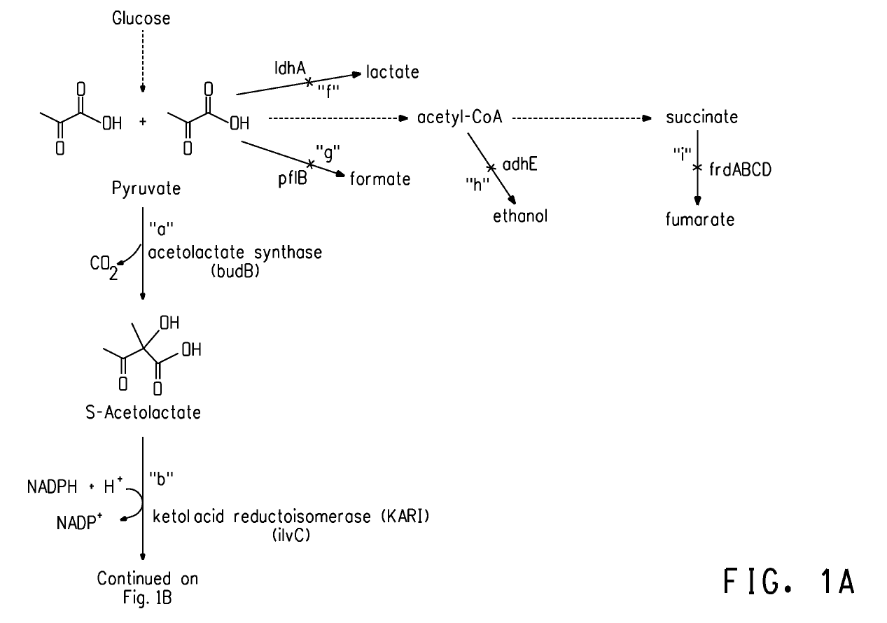

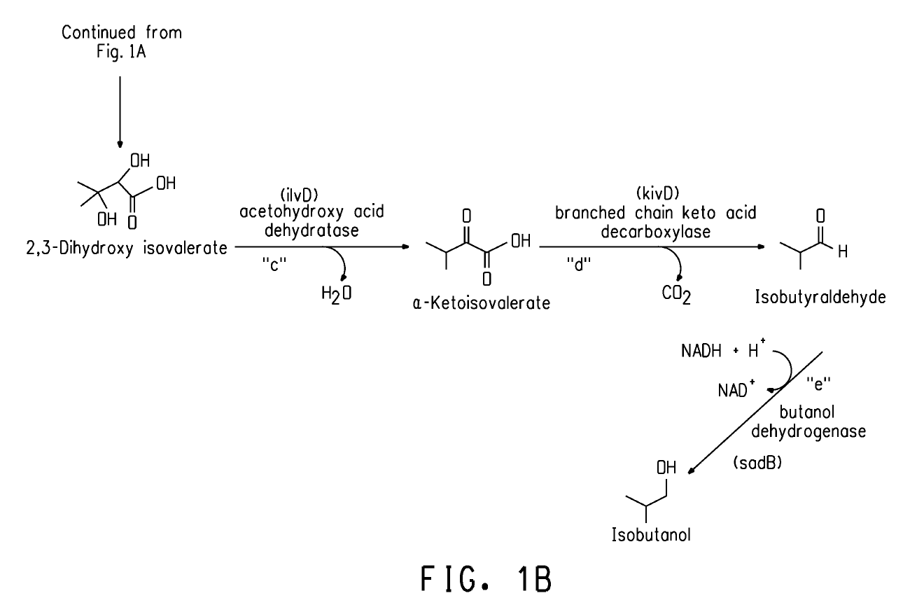

The present invention provides an enteric production host for isobutanol production comprising at least one gene encoding a polypeptide having secondary alcohol dehydrogenase activity wherein the host produces isobutanol and is substantially free of at least one of the following enzyme activities: pyruvate formate lyase, fumarate reductase enzyme complex, alcohol dehydrogenase and lactate dehydrogenase (see FIG. 1A, reactions “f”, “g”, “h”, “i”). The secondary alcohol dehydrogenase of the production host is particularly efficient in the conversion of isobutyraldehyde to isobutanol.

Deletion of lactate dehydrogenase (encoded by IdhA) prevents diversion of pyruvate for production of lactate (FIG. 1A, reaction “f”).

### Microbial Hosts for Isobutanol Production

The microbial hosts selected for isobutanol production should be able to convert carbohydrates to isobutanol. The criteria for selection of suitable microbial hosts include the following: high rate of glucose utilization, availability of genetic tools for gene manipulation, and the ability to generate stable chromosomal alterations.

Most microbes are capable of utilizing carbohydrates. However, certain environmental microbes cannot utilize carbohydrates with high efficiency, and therefore would not be suitable hosts.

The ability to genetically modify the host is essential for the production of any recombinant microorganism. The mode of gene transfer technology may be by electroporation, conjugation, transduction or natural transformation and are well known in the art. A broad range of host conjugative plasmids and drug resistance markers are available. The cloning vectors are tailored to the host microorganisms based on the nature of antibiotic resistance markers that can function in that host and are well known in the art.

The microbial host also has to be manipulated in order to inactivate competing pathways for carbon flow by deleting various genes as described herein below. Based on the criteria described above, the preferred hosts include various species of the genus: Escherichia, Salmonella, Klebsiella, Serratia, Erwinia and Shigella.

Due to toxicity of isobutanol to the microorganisms, it would also be desirable to either identify or engineer host strains that would be more tolerant to isobutanol. Selection of such tolerant hosts has been disclosed in a co-pending and commonly owned application US 20070259411.

### Creation of Knockout Mutants for Isobutanol Accumulation

Microorganisms metabolizing sugar substrates produce a variety of by-products in a mixed acid fermentation (Moat, A. G. et al., Microbial Physiology, 4^{th} edition, John Wiley Publishers, N.Y., 2002) Typical products of the mixed acid fermentation are acids such as formic, lactic and succinic acids and ethanol. Formation of these byproducts during an isobutanol fermentation can lower the potential yield of isobutanol. To prevent yield loss of isobutanol the enzyme activities corresponding to byproduct formation can be reduced. Enzymes involved in byproduct formation include, but are not limited to: 1) Pyruvate formate lyase (EC 2.3.1.54), encoded by pfIB gene (amino acid SEQ ID NO: 46; DNA SEQ ID NO: 47), that metabolizes pyruvate to formate and acetyl-coenzyme A. Deletion of this enzyme removes the competition for pyruvate to form formate and acetyl-CoA (FIG. 1A, reaction “g”);2) Fumarate reductase enzyme complex (EC 1.3.99.1), encoded by frdABCD operon, that catalyses the reduction of fumarate to succinate and requires NADH; the FrdA (amino acid SEQ ID NO: 54; DNA SEQ ID NO: 55) subunit contains a covalently bound flavin adenine dinucleotide.; FrdB contains the iron-sulfur centers of the enzyme (amino acid SEQ ID NO: 48; DNA SEQ ID NO: 49); FrdC (amino acid SEQ ID NO: 56; DNA SEQ ID NO: 57) and FrdD (amino acid SEQ ID NO: 58; DNA SEQ ID NO: 59) are integral membrane proteins that bind the catalytic FrdAB domain to the cytoplasmic mebrane. The function of fumarate reductase may be eliminated by deletion of any one of the subunits of frdA, B, C, or D, where deletion frdB is preferred. Deletion of this activity removes the draw for pyruvate for its conversion to fumarate (FIG. 1A, reaction “i”);3) Alcohol dehydrogenase (EC 1.2.1.10-acetaldehyde dehydrogenase and EC 1.1.1.1-alcohol dehydrogenase), encoded by adhE gene (amino acid SEQ ID NO: 52; DNA SEQ ID NO: 53), that synthesizes ethanol from acetyl-CoA in a two step reaction (both reactions are catalyzed by adhE and both reactions require NADH).(FIG. 1A, reaction “h”); and

4) Lactate dehydrogenase (EC 1.1.1.28), encoded by IdhA (amino acid SEQ ID NO: 50; DNA SEQ ID NO: 51) gene, that reduces pyruvate to lactate with oxidation of NADH. Deletion of this enzyme removes the competition for pyuruvate by this enzyme and blocks its conversion to formate and acetyl-CoA (FIG. 1A, reaction “g”). Methods for creating genetic mutations are common and well known in the art and may be applied to the exercise of creating mutants lacking pfIB (encoding for pyruvate formate lyase), frdB (encoding for a subunit of fumarate reductase), IdhA (encoding for lactate dehydrogenase) and adhE (encoding for alcohol dehydrogenase). Commonly used random genetic modification methods reviewed in Miller, J. H. (1992, A Short Course in Bacterial Genetics. Cold Spring Harbor Press, Plainview, N.Y.) include spontaneous mutagenesis, mutagenesis caused by mutator genes, chemical mutagenesis, irradiation with UV or X-rays, and transposon insertion. Transposons have been introduced into bacteria in a variety of ways including:
1. phage mediated transduction—has been used in both species specific and cross-species contexts.
2. conjugation—can be between members of the same or different species.
3. Transformation—chemically aided and electric shock mediated uptake of DNA can be used.

In these methods the transposon expresses a transposase in the recipient that catalyzes gene hopping from the incoming DNA to the recipient genome. The transposon DNA can be naked, incorporated in a phage or plasmid nucleic acid or complexed with a transposase. Most often the replication and/or maintenance of the incoming DNA containing the transposon is prevented, such that genetic selection for a marker on the transposon (most often antibiotic resistance).insures that each recombinant is the result of movement of the transposon from the entering DNA molecule to the recipient genome. An alternative method is one in which transposition is carried out with chromosomal DNA, fragments thereof or a fragment thereof in vitro, and then the novel insertion allele that has been created is introduced into a recipient cell where it replaces the resident allele by homologous recombination. Transposon insertion may be performed as described in Kleckner and Botstein (J. Mol. Biol., 116: 125-159, 1977) or as indicated above via any number of derivative methods.

A deletion of the pfIB, frdB, IdhA, adhE genes may also be constructed directly in the bacterial chromosome. The engineered chromosomal segments are inserted in the enteric bacterial target host chromosome at the site of the endogenous genes and replaces the endogenous region. Insertion of the engineered chromosomal segment may be by any method known to one skilled in the art, such as by phage transduction, conjugation, or plasmid introduction or non-plasmid double or single stranded DNA introduction followed by homologous recombination. In bacteriophage transduction, standard genetic methods for transduction are used which are well known in the art and are described by Miller, J. H. (Experiments in Molecular Genetics, Cold Spring Harbor Laboratory Press, Cold Spring Harbor, N.Y., 1972). The engineered chromosomal segment that has been constructed in a bacterial chromosome is packaged in the phage, then introduced to the target host cell through phage infection, followed by homologous recombination to insert the engineered chromosomal segment in the target host cell chromosome.

DNA fragments may be prepared from a bacterial chromosome bearing the engineered chromosomal segment by a method that includes sequences that naturally flank this chromosomal segment in the bacterial chromosome, to provide sequences where homologous recombination will occur. The flanking homologous sequences are sufficient to support homologous recombination, as described in Lloyd, R. G., and K. B. Low (Homologous recombination, p. 2236-2255; In F. C. Neidhardt, ed., Escherichia Coli and Salmonella: Cellular and Molecular Biology, 1996, ASM Press, Washington, DC). Typically homologous sequences used for homologous recombination are over 1 kb in length, but may be as short as 50 or 100 base pairs. DNA fragments containing the engineered chromosomal segment and flanking homologous sequences may be prepared with defined ends, such as by restriction digestion, or using a method that generates random ends such as sonication. In either case, the DNA fragments carrying the engineered chromosomal segment may be introduced into the target host cell by any DNA uptake method, including for example, electroporation, a freeze-thaw method, or using chemically competent cells. The DNA fragment undergoes homologous recombination which results in replacement of the endogenous chromosomal region of the target host with the engineered chromosomal segment.

A plasmid may be used to carry the engineered chromosomal segment into the target host cell for insertion. Typically a non-replicating plasmid is used to promote integration. The engineered chromosomal segment is flanked in the plasmid by DNA sequences that naturally flank this chromosomal segment in the bacterial target host genome, to provide sequences where homologous recombination will occur. The flanking homologous sequences are as described above and introduction of plasmid DNA is as described above.

Using any of these methods, homologous recombination may be enhanced by use of bacteriophage homologous recombination systems, such as the bacteriophage lambda Red system (Datsenko and Wanner, Proc. Natl. Acad. Sci. USA, 97: 6640-6645, 2000) and (Ellis et al., Proc. Natl. Acad. Sci. USA, 98: 6742-6746, 2001) or the Rac phage RecE/RecT system (Zhang et al., Nature Biotechnol., 18:1314-1317, 2000).

In any of these methods, the homologous recombination results in replacement of the endogenous chromosomal region of the target host with the engineered chromosomal segment.

Recipient strains with successful insertion of the engineered chromosomal segment may be identified using a marker. Either screening or selection markers may be used, with selection markers being particularly useful. For example, an antibiotic resistance marker may be present in the engineered chromosomal segment, such that when it is transferred to a new host, cells receiving the engineered chromosomal segment can be readily identified by growth on the corresponding antibiotic. Alternatively a screening marker may be used, which is one that confers production of a product that is readily detected. If it is desired that the marker not remain in the recipient strain, it may subsequently be removed such as by using site-specific recombination. In this case site-specific recombination sites are located 5′ and 3′ to the marker DNA sequence such that expression of the recombinase will cause deletion of the marker. Once the mutations have been created the cells must be screened for absence of these specific genes. A number of methods may be used to analyze for this purpose.

Any bacterial gene identified as pfIB, frdB, IdhA and adhE is a target for modification in the corresponding microorganism to create a strain of the present invention for production of isobutanol. The genes and gene products from various enteric microorganisms such as E. coli, Salmonella, Serratia, Erwinia, Shigella may be identified by hybridization, informatics or homologs as described herein.

### Isolation of Homologs

A nucleic acid molecule encoding genes of interest in the present invention such as SEQ ID NOs: 9, 46, 48, 50 and 52, or anyone of the sequences recited in the isobutanol biosynthetic pathway, described herein may be used to isolate nucleic acid molecules encoding homologous proteins, that have at least 70%-75%, 75%-80%, 80%-85%, 85%-90%, 90%-95%, or 95%-100% sequence identity to this nucleic acid fragment, from the same or other microbial species. Isolation of homologs using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., Polymerase Chain Reaction (PCR), Mullis et al., U.S. Pat. No. 4,683,202; Ligase Chain Reaction (LCR), Tabor, S. et al., (Proc. Natl. Acad. Sci. USA, 82: 1074, 1985); or Strand Displacement Amplification (SDA), (Walker, et al., Proc. Natl. Acad. Sci. USA, 89: 392, 1992).

For example, nucleic acid fragments of the instant invention may be isolated directly by using all or a portion of the nucleic acid fragment of SEQ ID NOs: 9, 46, 48, 50 and 52 as a DNA hybridization probe to screen libraries from any desired bacteria using methodology well known to those skilled in the art. Specific oligonucleotide probes based upon SEQ ID NOs: 9, 46, 48, 50 and 52 can be designed and synthesized by methods known in the art (Maniatis, supra). Moreover, the sequence can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or the full-length of homologs of the SEQ ID NOs: 9, 46, 48, 50 and 52. The resulting amplification products can be labeled directly during amplification reactions or labeled after amplification reactions, and used as probes to isolate full-length DNA fragments under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotide as specific hybridization probes in the Diagnosis of Genetic Disorders”, in Human Genetic Diseases: A Practical Approach, K. E. Davis Ed., (1986) pp. 33-50, IRL Press, Herndon, Va.); Rychlik, W., (1993) In White, B. A. (ed.), Methods in Molecular Biology, Vol. 15, pages 31-39, PCR Protocols: Current Methods and Applications, Humania Press, Inc., Totowa, N.J.).

Generally, two short segments of the instant nucleic acid sequence may be used to design primers for use in polymerase chain reaction protocols to amplify longer nucleic acid fragments encoding homologous coding regions from DNA or RNA. PCR may be performed using as template any DNA that contains a nucleic acid sequence homologous to SEQ ID NOs: 9, 46, 48,54, 56, 50 and 52, including for example, genomic DNA, cDNA or plasmid DNA as template. When using a library of cloned cDNA, the sequence of one primer is derived from SEQ ID NOs: 9, 46, 48,54, 56, 50 and 52, and the sequence of the other primer takes advantage of the presence of the polyadenylic acid tracts at the 3′ end of the mRNA precursor encoding microbial genes. Alternatively, the second primer sequence may be based upon sequences derived from the cloning vector. For example, the skilled artisan can follow the RACE protocol using mRNA as template (Frohman et al., Proc. Natl. Acad. Sci. USA, 85: 8998, 1988) to generate cDNAs by using PCR to amplify copies of the region between a single point in the transcript and the 3′ or 5′ end. Primers oriented in the 3′ and 5′ directions can be designed from the instant nucleic acid sequence. Using commercially available 3′ RACE or 5′ RACE systems (Life Technologies, Rockville, Md.), specific 3′ or 5′ cDNA fragments can be isolated (Ohara et al., Proc. Natl. Acad. Sci. USA, 86: 5673, 1989); and (Loh et al., Science, 243: 217, 1989).

### Hybridization

Alternatively nucleic acid molecules of SEQ ID NOs: 9, 46, 48,54, 56, 50 and 52, or their complements may be employed as a hybridization reagent for the identification of homologs. The basic components of a nucleic acid hybridization test include a probe, a sample suspected of containing the gene or gene fragment of interest, and a specific hybridization method. Probes of the present invention are typically single stranded nucleic acid sequences which are complementary to the nucleic acid sequences to be detected. Probes are “hybridizable” to the nucleic acid sequence to be detected. The probe length may vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151, 1991). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions may be employed. Typically, these comprise from about 20 to 60% volume, preferably 30%, of a polar organic solvent. A common hybridization solution employs about 30-50% v/v formamide, about 0.15 to 1M sodium chloride, about 0.05 to 0.1M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kD), polyvinylpyrrolidone (about 250-500 kD), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% w/v glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate.

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

In addition, since sequences of microbial genomes are rapidly becoming available to the public, homologs may be identified using bioinformatics approaches alone.

Accordingly the invention provides recombinant enteric bacterial cells wherein the genetic modification results in deletion of specific pfIB, frdB, IdhA and adhE genes to allow focused flow of the carbon to isobutanol production.

### Isobutanol Biosynthetic Pathways

Carbohydrate utilizing microorganisms employ the Embden-Meyerhof-Parnas (EMP) pathway, the Entner-Doudoroff pathway and the pentose phosphate cycle as the central, metabolic routes to provide energy and cellular precursors for growth and maintenance. These pathways have in common the intermediate glyceraldehyde-3-phosphate and, ultimately, pyruvate is formed directly or in combination with the EMP pathway. Subsequently, pyruvate is transformed to acetyl-coenzyme A (acetyl-CoA) via a variety of means. Acetyl-CoA serves as a key intermediate, for example, in generating fatty acids, amino acids and secondary metabolites. The combined reactions of sugar conversion to pyruvate produce energy (e.g. adenosine-5′-triphosphate, ATP) and reducing equivalents (e.g. reduced nicotinamide adenine dinucleotide, NADH, and reduced nicotinamide adenine dinucleotide phosphate, NADPH). NADH and NADPH must be recycled to their oxidized forms (NAD⁺ and NADP⁺, respectively). In the presence of inorganic electron acceptors (e.g. O₂, NO₃⁻ and SO₄²⁻), the reducing equivalents may be used to augment the energy pool; alternatively, a reduced carbon by-product may be formed.

The enteric host of the invention produces isobutanol. Typically an isobutanol biosynthetic pathway will be engineered into the host cell that will enable the host cell to produce isobutanol from carbohydrates as shown in FIGS. 1A and 1B. One pathway comprises the following substrate to product conversions:
a) pyruvate to acetolactate, as catalyzed by acetolactate synthase,
b) acetolactate to 2,3-dihydroxyisovalerate, as catalyzed by acetohydroxy acid isomeroreductase,
c) 2,3-dihydroxyisovalerate to α-ketoisovalerate, as catalyzed by acetohydroxy acid dehydratase,
d) α-ketoisovalerate to isobutyraldehyde, as catalyzed by a branched-chain keto acid decarboxylase, and
e) isobutyraldehyde to isobutanol, as catalyzed by a butanol dehydrogenase (secondary alcohol dehydrogenase).

This pathway combines enzymes known to be involved in the well-characterized pathways for valine biosynthesis (pyruvate to α-ketoiso-valerate) and valine catabolism (α-ketoisovalerate to isobutyraldehyde) and the final step of a novel butanol dehydrogenase. Alternate isobutantol pathways are described in commonly owned and co-pending US Application 20070092957, incorporated herein by reference.

Since many valine biosynthetic enzymes also catalyze analogous reactions in the isoleucine biosynthetic pathway, substrate specificity is a major consideration in selecting the gene sources. The primary genes of interest therefore for the acetolactate synthase enzyme are those from Bacillus (alsS) and Klebsiella (budB). These particular acetolactate synthases are known to participate in butanediol fermentation in these microorganisms and show increased affinity for pyruvate over ketobutyrate (Gollop et al., J. Bacteriol. 172: 3444-3449, 1990); Holtzclaw et al., J. Bacteriol. 121: 917-922, 1975). The second and third pathway steps are catalyzed by acetohydroxy acid reductoisomerase and dehydratase, respectively. These enzymes have been characterized from a number of sources, such as for example, E. coli (Chunduru et al., Biochemistry 28:486-493, 1989; and Flint et al., J. Biol. Chem. 268:14732-14742, 1993). The final two steps of the preferred isobutanol pathway are known to occur in yeast, which can use valine as a nitrogen source and, in the process, secrete isobutanol. α-Ketoisovalerate may be converted to isobutyraldehyde by a number of keto acid decarboxylase enzymes, such as for example pyruvate decarboxylase. To prevent misdirection of pyruvate away from isobutanol production, a decarboxylase with decreased affinity for pyruvate is desired. So far, there are two such enzymes known in the art (Smit et al., Appl. Environ. Microbiol. 71: 303-311, 2005; and de la Plaza et al., FEMS Microbiol. Lett. 238: 367-374, 2004). Both enzymes are from strains of Lactococcus lactis and have a 50-200-fold preference for ketoisovalerate over pyruvate. Finally, a number of aldehyde reductases have been identified in yeast, many with overlapping substrate specificity. Those known to prefer branched-chain substrates over acetaldehyde include, but are not limited to, alcohol dehydrogenase VI (ADH6) and Ypr1p (Larroy et al., Biochem. J. 361: 163-172, 2002; and Ford et al., Yeast 19: 1087-1096, 2002), both of which use NADPH as electron donor. An NADPH-dependent reductase, YqhD, active with branched-chain substrates has also been recently identified in E. coli (Sulzenbacher et al., J. Mol. Biol. 342: 489-502, 2004).

In the isobutanol pathway of the current disclosure, a novel butanol dehydrogenase from Achromobacter xylosoxidans is used and is described herein.

Butanol Dehydrogenase Activity of Achromobacter xylosoxidans

Through enriching an environmental sludge sample by serially culturing on medium containing 1-butanol, microorganisms were isolated that are capable of using 1-butanol as a sole carbon source. One isolate was identified by its 16S rRNA sequence as belonging to the bacterial species Achromobacter xylosoxidans. This isolate contains a butanol dehydrogenase enzyme activity which interconverted butyraldehyde and 1-butanol. Unexpectedly it was found that this butanol dehydrogenase enzyme activity also catalyzed the interconversion of isobutyraldehyde and isobutanol, as well as the interconversion of 2-butanone and 2-butanol. Surprisingly, this enzyme had kinetic constants for the alternate substrates comparable or superior to that for the 1-butanol substrate used in the enriching medium. These results indicated that this Achromobacter xylosoxidans butanol dehydrogenase may be used for production of 1-butanol, isobutanol, or 2-butanol in a recombinant microbial host cell having a source of the butyraldehyde, isobutyraldehyde or 2-butanone substrate, respectively.

### Butanol Dehydrogenase Protein and Coding Sequence

The nucleotide sequence identified in Achromobacter xylosoxidans that encodes an enzyme with butanol dehydrogenase activity is given as SEQ ID NO: 9. The amino acid sequence of the full protein is given as SEQ ID NO:10. Comparison of this amino acid sequence to sequences in public databases revealed that this protein has surprisingly low similarity to known alcohol dehydrogenases. The most similar known sequences are 67% identical to the amino acid sequence of SEQ ID NO:10 over its length of 348 amino acids using BLAST with scoring matrix BLOSUM62, an expect cutoff of 10 and word size 3. A gap opening penalty of 11 and a gap extension of 1 were used. The closest similarities found were 67% amino acid identity to a Zn-containing alcohol dehydrogenase of Neisseria meningitides MC58 (Accession #AAF41759.1) and 67% amino acid identity to the Zn-containing alcohol dehydrogenase of Mycoplasma agalactiae (Accession #A5IY63). Thus preferred butanol dehydrogenases (sadB) are those that are at least about 70%-75%, about 75%-80%, about 80%-85%, 85%-90%, or 90%-95% identical to SEQ ID NO: 10 over its length of 348 amino acids using BLAST with scoring matrix BLOSUM62, an expect cutoff of 10 and word size 3 and a gap opening penalty of 11 and a gap extension of 1.

### Butanol Dehydrogenase Activity

Proteins that have at least about 70% or greater amino acid identity to SEQ ID NO: 10 and have butanol dehydrogenase activity are particularly useful in the present invention. Nucleic acid molecules of the invention encode proteins with at least about 70% or greater amino acid identity to SEQ ID NO: 10 having butanol dehydrogenase activity. One skilled in the art can readily assess butanol dehyrogenase activity in a protein. A protein is expressed in a microbial cell as described below and assayed for butanol dehydrogenase activity in cell extracts, crude enzyme preparations, or purified enzyme preparations. For example, assay of purified enzyme and crude enzyme preparations are described in Example 1 herein. An assay for 1-butanol dehydrogenase activity monitors the disappearance of NADH spectrophotometrically at 340 nm using appropriate amounts of enzyme in 50 mM potassium phosphate buffer, pH 6.2 at 35° C. containing 50 mM butyraldehyde and 0.2 mM NADH. An alternative assay with an alcohol substrate is performed at 35° C. in TRIS buffer, pH 8.5, containing 3 mM NAD⁺ and varying concentrations of alcohol, or with a ketone or aldehyde substrate is performed at 35° C. with 50 mM MES buffer, pH 6.0, 200 μM NADH and varying concentrations of the ketone or aldehyde. Through these or other readily performable assays butanol dehydrogenase function is linked to structure of an identified protein encoded by an isolated nucleic acid molecule, both of which have an identified sequence.

### Construction of Production Host

Recombinant microorganisms containing the necessary genes that will encode the enzymatic pathway for the conversion of a fermentable carbon substrate to isobutanol may be constructed using techniques well known in the art. Genes encoding the enzymes of the isobutanol biosynthetic pathways of the invention, i.e., acetolactate synthase, ketol acid reductoisomerase, acetohydroxy acid dehydratase, branched-chain α-keto acid decarboxylase, and branched-chain alcohol dehydrogenase, may be isolated from various sources, as described above.

The construction of the isobutanol producing strain used for manipulations disclosed in this application has been disclosed in the commonly owned and co-pending US Application 20070092957. In particular Examples 1, 2, 9, 10, 11, 12, 13 and 14 which is incorporated herein by reference.

Methods of obtaining desired genes from a microbial genome well known in the art of molecular biology. For example, if the sequence of the gene is known, suitable genomic libraries may be created by restriction endonuclease digestion and may be screened with probes complementary to the desired gene sequence. Once the sequence is isolated, the DNA may be amplified using standard primer-directed amplification methods such as polymerase chain reaction (U.S. Pat. No. 4,683,202) to obtain amounts of DNA suitable for transformation using appropriate vectors. Tools for codon optimization for expression in a heterologous host are readily available. Some tools for codon optimization are available based on the GC content of the host microorganism.

Once the relevant pathway genes are identified and isolated they may be transformed into suitable expression hosts by means well known in the art. Vectors or cassettes useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IP_{L}, IP_{R}, T7, tac, and trc (useful for expression in Escherichia coli and other Enterobacteriaceae).

Termination control regions may also be derived from various genes native to the preferred hosts. Optionally, a termination site may be unnecessary, however, it is most preferred if included.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid, 50: 74-79, 2003). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

The expression of an isobutanol biosynthetic pathway in various preferred microbial hosts is described in more detail below.

Expression of an Isobutanol Biosynthetic Pathway in E. coli

Vectors or cassettes useful for the transformation of E. coli are common and commercially available from the companies listed above. For example, the genes of an isobutanol biosynthetic pathway may be isolated from various sources, cloned into a modified pUC19 vector and transformed into E. coli.

### Expression of the Isobutanol Biosynthetic Pathway in the Family of Enterobacteriaceae

Examples of enteric bacteria suitable for use in this invention include, but not limited to, members of the genus Serratia, Erwinia, Escherichia, Klebsiella, Salmonella, and Shigella. Methods for gene expression and creation of mutations in Enterobacteriaceae are also well known in the art. For example, the genes of an isobutanol biosynthetic pathway may be isolated from various sources, cloned into various vectors as described in Examples 1, 2, 9, 19, 11, 12, 13 and 14 of the commonly owned and co-pending US Application 20070092957. Particularly suitable in the present invention are members of the enteric class of bacteria. Enteric bacteria are members of the family Enterobacteriaceae and include such members as Escherichia, Salmonella, and Shigella. They are gram-negative straight rods, 0.3-1.0×1.0-6.0 mm, motile by peritrichous flagella (except for Tatumella) or nonmotile. They grow in the presence and absence of oxygen and grow well on various media such as peptone, meat extract, and (usually) MacConkey's. Some grow on D-glucose as the sole source of carbon, whereas others require vitamins and/or mineral(s). They are chemoorganotrophic with respiratory and fermentative metabolism but are not halophilic. Acid and often visible gas is produced during fermentation of D-glucose, other carbohydrates, and polyhydroxyl alcohols. They are oxidase negative and, with the exception of Shigella dysenteriae 0 group 1 and Xenorhabdus nematophilus, catalase positive. Nitrate is reduced to nitrite (except by some strains of Erwinia and Yersina). The G+C content of DNA is 38-60 mol % (Tm, Bd). DNAs from species within most genera are at least 20% related to one another and to Escherichia coli, the type species of the family. Notable exceptions are species of Yersina, Proteus, Providenica, Hafnia and Edwardsiella, whose DNAs are 10-20% related to those of species from other genera. Except for Erwinia chrysanthemi, all species tested contain the enterobacterial common antigen (Bergy's Manual of Systematic Bacteriology, D. H. Bergy et al., Williams and Wilkins Press, Baltimore, 1984).

### Fermentable Carbon Substrates

Recombinant microbial production host of the present invention must contain suitable carbon substrates. Suitable carbon substrates may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt.

Although it is contemplated that all of the above mentioned carbon substrates and mixtures thereof are suitable in the present invention, preferred carbon substrates are glucose, fructose, and sucrose. Sucrose may be derived from renewable sugar sources such as sugar cane, sugar beets, cassava, sweet sorghum, and mixtures thereof. Glucose (dextrose) may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass through processes of pretreatment and saccharification, as described, for example, in commonly owned and co-pending US Patent Application Publication No. 20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In addition to an appropriate carbon substrate, fermentation medium must contain suitable minerals, salts, cofactors, buffers and other components, known to those skilled in the art, suitable for the growth of the cultures and promotion of the enzymatic pathway necessary for isobutanol production.

### Culture Conditions

Typically cells are grown at a temperature in the range of about 25° C. to about 40° C. in an appropriate medium. Suitable growth media in the present invention are common commercially prepared media such as Luria Bertani (LB) broth. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation are between pH 5.0 to pH 9.0, where pH 6.0 to pH 8.0 is preferred as the initial condition.

Fermentations may be performed under aerobic or anaerobic conditions, where anaerobic or microaerobic conditions are preferred.

The amount of isobutanol produced in the fermentation medium may be determined using a number of methods known in the art, for example, high performance liquid chromatography (HPLC) or gas chromatography (GC).

### Industrial Batch and Continuous Fermentations

A batch method of fermentation may be used. A classical batch fermentation is a closed system where the composition of the medium is set at the beginning of the fermentation and not subject to artificial alterations during the fermentation. Thus, at the beginning of the fermentation the medium is inoculated with the desired microorganism(s), and fermentation is permitted to occur without adding anything to the system. Typically, however, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Thomas D. Brock in (Biotechnology: A Textbook of Industrial Microbiology, Second Edition, 1989, Sinauer Associates, Inc., Sunderland, Mass.), or in Deshpande, Mukund V., (Appl. Biochem. Biotechnol., 36:227, 1992), herein incorporated by reference.

Although the present invention is performed in batch mode it is contemplated that the method would be adaptable to continuous fermentation methods. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by the medium turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for isobutanol production.

Methods for Isobutanol Isolation from the Fermentation Medium

The bioproduced isobutanol may be isolated from the fermentation medium using methods known in the art. For example, solids may be removed from the fermentation medium by centrifugation, filtration, decantation, or the like. Then, the isobutanol may be isolated from the fermentation medium, which has been treated to remove solids as described above, using methods such as distillation, liquid-liquid extraction, or membrane-based separation. Because isobutanol forms a low boiling point, azeotropic mixture with water, distillation may only be used to separate the mixture up to its azeotropic composition. Distillation may be used in combination with another separation method to obtain separation around the azeotrope. Methods that may be used in combination with distillation to isolate and purify isobutanol include, but are not limited to, decantation, liquid-liquid extraction, adsorption, and membrane-based techniques. Additionally, isobutanol may be isolated using azeotropic distillation using an entrainer (see for example Doherty and Malone, Conceptual Design of Distillation Systems, McGraw Hill, New York, 2001).

The isobutanol-water mixture forms a heterogeneous azeotrope so that distillation may be used in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase may be returned to the first distillation column as reflux. The isobutanol-rich decanted organic phase may be further purified by distillation in a second distillation column.

The isobutanol may also be isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the isobutanol from the solvent.

Distillation in combination with adsorption may also be used to isolate isobutanol from the fermentation medium. In this method, the fermentation broth containing the isobutanol is distilled to near the azeotropic composition and then the remaining water is removed by use of an adsorbent, such as molecular sieves (Aden et al. Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245:199-210, 2004).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques used in the Examples are well known in the art and are described by Sambrook, J., et al., in Molecular Cloning: A Laboratory Manual; (Cold Spring Harbor Laboratory Press: Cold Spring Harbor, N.Y., 1989, also known as Maniatis) and by T. J. Silhavy, M. L. Bennan, and L. W. Enquist in Experiments with Gene Fusions (Cold Spring Harbor Laboratory Press, Cold Spring Harbor, N.Y.,1984) and by Ausubel, F. M. et al., in Current Protocols in Molecular Biology, (Greene Publishing Assoc. and Wiley-Interscience, N.Y., 1987).

Materials and methods suitable for the maintenance and growth of bacterial cultures are well known in the art. Techniques suitable for use in the following Examples may be found as set out in Manual of Methods for General Bacteriology (Phillipp Gerhardt, et al., eds., American Society for Microbiology, Washington, D.C., 1994) or by Thomas D. Brock in Biotechnology: A Textbook of Industrial Microbiology, (2^{nd} Edition, Sinauer Associates, Inc., Sunderland, Mass., 1989). All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.) unless otherwise specified.

Microbial strains were obtained from The American Type Culture Collection (ATCC), Manassas, Va., unless otherwise noted.

### P1 Transduction

P1ᵥᵢᵣ transductions were carried out as described by Miller with some modifications (Miller, J. H. 1992. A Short Course in Bacterial Genetics. Cold Spring Harbor Laboratory, Cold Spring Harbor, N.Y). Briefly, to prepare a transducing lysate, cells of the donor strain were grown overnight in the Luria Broth (LB) medium at 37° C. while shaking. An overnight growth of these cells was sub-cultured into the LB medium containing 0.005M CaCl₂. and placed in a 37° C. water bath with no aeration. One hour prior to adding phage, the cells were placed at 37° C. with shaking. After final growth of the cells, a 1.0 mL aliquot of the culture was dispensed into 14 ml Falcon tubes and approximately 10⁷ P1ᵥᵢᵣ phage was added. These tubes were incubated in a 37° C. water bath for 20 min before 2.5 mL of 0.8% LB top agar was added to each tube, the contents were spread on an LB agar plate and were incubated at 37° C. The following day the soft agar layer was scraped into a centrifuge tube. The surface of the plate was washed with the LB medium and added to the centrifuge tube followed by a few drops of CHCl₃ before the tube was vigorously agitated using a Vortex mixer. After centrifugation at 4,000 rpm for 10 min, the supernatant containing the P1ᵥᵢᵣ lysate was collected.

For transduction, the recipient strain was grown overnight in 1-2 mL of the LB medium at 37° C. with shaking. Cultures were pelleted by centrifugation in an Eppendorf Microcentrifuge at 10,000 rpm for 1 min at room temp. The cell pellet was resuspended in an equal volume of MC buffer (0.1 M MgSO₄, 0.005 M CaCl₂), dispensed into tubes in 0.1 mL aliquots and 0.1 mL and 0.01 mL of P1ᵥᵢᵣ lysate was added. A control tube containing no P1ᵥᵢᵣ lysate was also included. Tubes were incubated for 20 min at 37° C. before 0.2 mL of 0.1 M sodium citrate was added to stop the P1 infection. One mL of the LB medium was added to each tube before they were incubated at 37° C. for 1 hr. After incubation the cells were pelleted as described above, resuspended in 50-200 μL of the LB prior to spreading on the LB plates containing 25 μg/mL kanamycin and were incubated overnight at 37° C. Transductants were screened by colony PCR with chromosome specific primers flanking the region upstream and downstream of the kanamycin marker insertion.

Removal of the kanamycin marker from the chromosome was obtained by transforming the kanamycin-resistant strain with plasmid pCP20 (Cherepanov, P. P. and Wackernagel, W., Gene, 158: 9-14, 1995) followed by spreading onto the LB ampicillin (100 μg/mL) plates and incubating at 30° C. The pCP20 plasmid carries the yeast FLP recombinase under the control of the λ_{PR} promoter. Expression from this promoter is controlled by the c1857 temperature-sensitive repressor residing on the plasmid. The origin of replication of pCP20 is also temperature-sensitive. Ampicillin resistant colonies were streaked onto the LB agar plates and incubated at 42° C. The higher incubation temperature simultaneously induced expression of the FLP recombinase and cured the pCP20 plasmid from the cell. Isolated colonies were patched to grids onto the LB plates containing kanamycin (25 μg/mL), and LB ampicillin (100 μg/mL) plates and LB plates. The resulting kanamycin-sensitive, ampicillin-sensitive colonies were screened by colony PCR to confirm removal of the kanamycin marker from the chromosome.

For colony PCR amplifications the HotStarTaq Master Mix (Qiagen, Valencia, Calif.; catalog no. 71805-3) was used according to the manufacturer's protocol. Into a 25 μL Master Mix reaction containing 0.2 μM of each chromosome specific PCR primer, a small amount of a colony was added. Amplification was carried out in a DNA Thermocycler GeneAmp 9700 (PE Applied Biosystems, Foster City, Calif.). Typical colony PCR conditions were: 15 min at 95° C.; 30 cycles of 95° C. for 30 sec, annealing temperature ranging from 50-58° C. for 30 sec, primers extended at 72° C. with an extension time of approximately 1 min/kb of DNA; then 10 min at 72° C. followed by a hold at 4° C. PCR product sizes were determined by gel electrophoresis by comparison with known molecular weight standards.

Restriction enzymes, T4 DNA ligase and Phusion High Fidelity DNA Polymerase (New England Biolabs, Beverely, Mass.) were used according to manufacturer's recommendation.

Gel electrophoresis was done using the RunOne electrophoresis system (Embi Tec, San Diego, Calif.) with precast Reliant® 1 % agarose gels (Lonza Rockland, Inc. Rockland, Me.) according to manufacturer's protocols. Gels are typically run in TBE buffer (Invitrogen, Cat. No. 15581-044).

For transformations, electrocompetent cells of E. coli were prepared as described by Ausubel, F. M., et al., (Current Protocols in Molecular Biology, 1987, Wiley-Interscience,). Cells were grown in 25-50 mL the LB medium at 30-37° C. and harvested at an OD₆₀₀ of 0.5-0.7 by centrifugation at 10,000 rpm for 10 min. These cells are washed twice in sterile ice-cold water in a volume equal to the original starting volume of the culture. After the final wash cells were resuspended in sterile water and the DNA to be transformed was added. The cells and DNA were transferred to chilled cuvettes and electroporated in a Bio-Rad Gene Pulser II according to manufacturer's instructions (Bio-Rad Laboratories, Inc., Hercules, Calif.).

The oligonucleotide primers to use in the following Examples are given in Table 2. All the oligonucleotide primers were synthesized by Sigma-Genosys (Woodlands, Tex.).

### Methods for Determining Isobutanol Concentration in the Culture Medium

The concentration of isobutanol in the aqueous phase and organic phase was determined by gas chromatography (GC) using an HP-InnoWax column (30 m×0.32 mm ID, 0.25 μm film) from Agilent Technologies (Santa Clara, Calif.). The carrier gas was helium at a flow rate of 1 mL/min measured at 150° C. with constant head pressure; injector split was 1:10 at 200° C.; oven temperature was 45° C. for 1 min, 45° C. to 230° C. at 10° C./min, and 230° C. for 30 sec. Flame ionization detection was used at 260° C. with 40 mL/min helium makeup gas. Culture broth samples were filtered through 0.2 μm spin filters before injection into GC. Depending on the analytical sensitivity desired, either 0.1 μL or 0.5 μL injection volumes were used. Calibrated standard curves were generated for the following compounds: ethanol, isobutanol, acetoin, meso-2,3-but-anediol, and (2S,3S)-2,3-butanediol. Analytical standards were also used to identify retention times for isobutryaldehyde, isobutyric acid, and isoamyl alcohol. Under these conditions, the isobutanol retention time was about 5.33 minutes.

The meaning of abbreviations is as follows: “m” means meter, “mm” means millimeter, “μm” means microns or micro meter, “sec” means second(s), “min” means minute(s), “hr” means hour(s), “nm” means nanometers, “μL” means microliter(s), “mL” means milliliter(s), “rpm” means revolution per minute, “L” means liter(s), “mm” means millimeter(s), “nm” means nanometers, “mM” means millimolar, “μg/mL” means microgram per milliliter, “mmol/min/mg” means millimole per minute per milligram, “μM” means micromolar, “M” means molar, “mmol” means millimole(s), “μmol” means micromole(s), “g” means gram(s), “μg” means microgram(s), “PCR” means polymerase chain reaction, “OD” means optical density, “OD₆₀₀” means the optical density measured at a wavelength of 600 nm, “kD” means kilodaltons, “bp” means base pair(s), “kb” means killo base pair, “%” means percent, “% w/v” means weight/volume percent, “% v/v” means volume/volume percent, “IPTG” means isopropyl-β-D-thiogalactopyranoiside, “wt %” means weight percent, “RBS” means ribosome binding site, “HPLC” means high performance liquid chromatography, “GC” means gas chromatography, “g/L” means gram per liter, “g/L/h” means gram per liter per hour, and “g/g” means gram per gram, “mL/min” means milliliter per minute, “° C./min” means degrees Celsius per minute, “vvm” means volume to volume per minute, “v/v” means volume for volume, “vol %” means volume percent, “ID” means internal diameter.

### Example 1

### Construction of an E. coli Strain Having Deletions of pfIB, frdB, IdhA, and adhE Genes

This example describes engineering of an E. coli strain in which four genes were inactivated. The Keio collection of E. coli strains (Baba et al., Mol. Syst. Biol., 2:1-11, 2006) was used for production of the 4KO E. coli (four-knock out). The Keio collection is a library of single gene knockouts created in strain E. coli BW25113 by the method of Datsenko and Wanner (Datsenko, K. A. & Wanner, B. L., Proc Natl Acad Sci., USA, 97: 6640-6645, 2000). In the collection, each deleted gene was replaced with a FRT-flanked kanamycin marker that was removable by Flp recombinase. The 4KO E. coli strain was constructed by moving the knockout-kanamycin marker from the Keio donor strain by P1 transduction to a recipient strain. After each P1 transduction to produce a knockout, the kanamycin marker was removed by Flp recombinase. This markerless strain acted as the new donor strain for the next P1 transduction.

The 4KO E. coli strain was constructed in the Keio strain JW0886 by P1ᵥᵢᵣ transductions with P1 phage lysates prepared from three Keio strains in addition to JW0886. The Keio strains used are listed below:

JW0886: the kan marker is inserted in the pfIB

JW4114: the kan marker is inserted in the frdB

JW1375: the kan marker is inserted in the IdhA

JW1228: the kan marker is inserted in the adhE

Removal of the kanamycin marker from the chromosome was performed by transforming the kanamycin-resistant strain with pCP20 an ampicillin-resistant plasmid (Cherepanov,and Wackernagel, supra)). Transformants were spread onto LB plates containing 100 μg/mL ampicillin. Plasmid pCP20 carries the yeast FLP recombinase under the control of the λ_{PR} promoter and expression from this promoter is controlled by the cI857 temperature-sensitive repressor residing on the plasmid. The origin of replication of pCP20 is also temperature-sensitive.

Strain JW0886 (ΔpfIB::kan) was transformed with plasmid pCP20 and spread on the LB plates containing 100 μg/mL ampicillin at 30° C. Ampicillin resistant transformants were then selected, streaked on the LB plates and grown at 42° C. Isolated colonies were patched onto the ampicillin and kanamycin selective medium plates and LB plates. Kanamycin-sensitive and ampicillin-sensitive colonies were screened by colony PCR with primers pfIB CkUp (SEQ ID NO: 34) and pfIB CkDn (SEQ ID NO: 35). A 10 μL aliquot of the PCR reaction mix was analyzed by gel electrophoresis. The expected approximate 0.4 kb PCR product was observed confirming removal of the marker and creating the “JW0886 markerless” strain. This strain has a deletion of the pfIB gene.

The “JW0886 markerless” strain was transduced with a P1ᵥᵢᵣ lysate from JW4114 (frdB::kan) and streaked onto the LB plates containing 25 μg/mL kanamycin. The kanamycin-resistant transductants were screened by colony PCR with primers frdB CkUp (SEQ ID NO: 36) and frdB CkDn (SEQ ID NO: 37). Colonies that produced the expected approximate 1.6 kb PCR product were made electrocompetent and transformed with pCP20 for marker removal as described above. Transformants were first spread onto the LB plates containing 100 μg/mL ampicillin at 30° C. and ampicillin resistant transformants were then selected and streaked on LB plates and grown at 42° C. Isolated colonies were patched onto ampicillin and the kanamycin selective medium plates and LB plates. Kanamycin-sensitive, ampicillin-sensitive colonies were screened by PCR with primers frdB CkUp (SEQ ID NO: 36) and frdB CkDn (SEQ ID NO: 37). The expected approximate 0.4 kb PCR product was observed confirming marker removal and creating the double knockout strain, “ΔpfIB frdB”.

The double knockout strain was transduced with a P1ᵥᵢᵣ lysate from JW1375 (ΔIdhA::kan) and spread onto the LB plates containing 25 μg/mL kanamycin. The kanamycin-resistant transductants were screened by colony PCR with primers IdhA CkUp (SEQ ID NO: 38) and IdhA CkDn (SEQ ID NO: 39). Clones producing the expected 1.1 kb PCR product were made electrocompetent and transformed with pCP20 for marker removal as described above. Transformants were spread onto LB plates containing 100 μg/mL ampicillin at 30° C. and ampicillin resistant transformants were streaked on LB plates and grown at 42° C. Isolated colonies were patched onto ampicillin and kanamycin selective medium plates and LB plates. Kanamycin-sensitive, ampicillin-sensitive colonies were screened by PCR with primers IdhA CkUp (SEQ ID NO: 38) and IdhA CkDn (SEQ ID NO: 39) for a 0.3 kb product. Clones that produced the expected approximate 0.3 kb PCR product confirmed marker removal and created the triple knockout strain designated “3KO” (ΔpfIB frdB IdhA).

Strain “3 KO” was transduced with a P1ᵥᵢᵣ lysate from JW1228 (ΔadhE::kan) and spread onto the LB plates containing 25 μg/mL kanamycin. The kanamycin-resistant transductants were screened by colony PCR with primers adhE CkUp (SEQ ID NO: 40) and adhE CkDn (SEQ ID NO: 41). Clones that produced the expected 1.6 kb PCR product were made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers adhE CkUp (SEQ ID NO: 40) and adhE CkDn (SEQ ID NO: 41). Clones that produced the expected approximate 0.4 kb PCR product were named “4KO” (ΔpfIB frdB IdhA adhE).

### Example 2

### Construction of an E. coli Production Host Containing an Isobutanol Biosynthetic Pathway and Deletions of pfIB, frdB, IdhA, and adhE Genes

A DNA fragment encoding a butanol dehydrogenase (DNA SEQ ID NO:9; protein SEQ ID NO: 10) from Achromobacter xylosoxidans was amplified from A. xylosoxidans genomic DNA using standard conditions. The DNA was prepared using a Gentra Puregene kit (Gentra Systems, Inc., Minneapolis, Minn.; catalog number D-5500A) following the recommended protocol for gram negative microorganisms. PCR amplification was done using forward and reverse primers N473 and N469 (SEQ ID NOs: 44 and 45), respectively with Phusion high Fidelity DNA Polymerase (New England Biolabs, Beverly, Mass.). The PCR product was TOPO-Blunt cloned into pCR4 BLUNT (Invitrogen) to produce pCR4Blunt::sadB, which was transformed into E. coli Mach-1 cells. Plasmid was subsequently isolated from four clones, and the sequence verified.

The sadB coding region was then cloned into the vector pTrc99a (Amann et al., Gene 69: 301-315, 1988). The pCR4Blunt::sadB was digested with EcoRI, releasing the sadB fragment, which was ligated with EcoRI-digested pTrc99a to generate pTrc99a::sadB. This plasmid was transformed into E. coli Mach 1 cells and the resulting transformant was named Mach1/pTrc99a::sadB. The activity of the enzyme expressed from the sadB gene in these cells was determined to be 3.5 mmol/min/mg protein in cell-free extracts when analyzed using isobutyraldehyde as the standard.

The sadB gene was then subcloned into pTrc99A::budB-ilvC-ilvD-kivD as described below. The pTrc99A::budB-ilvC-ilvD-kivD is the pTrc-99a expression vector carrying an operon for isobutanol expression (described in Examples 9-14 the of the co-pending and commonly owned US Application 20070092957, which are incorporated herein by reference). The first gene in the pTrc99A::budB-ilvC-ilvD-kivD isobutanol operon is budB encoding encoding acetolactate synthase from Klebsiella pneumoniae ATCC 25955, followed by the ilvC gene encoding acetohydroxy acid reductoisomerase from E. coli. This is followed by ilvD encoding acetohydroxy acid dehydratase from E. coli and lastly the kivD gene encoding the branched-chain keto acid decarboxylase from L. lactis.

The sadB coding region was amplified from pTrc99a::sadB using primers N695A (SEQ ID NO: 42) and N696A (SEQ ID NO: 43) with Phusion High Fidelity DNA Polymerase (New England Biolabs, Beverly, Mass.). Amplification was carried out with an initial denaturation at 98° C. for 1 min, followed by 30 cycles of denaturation at 98° C. for 10 sec, annealing at 62° C. for 30 sec, elongation at 72° C. for 20 sec and a final elongation cycle at 72° C. for 5 min, followed by a 4° C. hold. Primer N695A contained an AvrII restriction site for cloning and a RBS upstream of the ATG start codon of the sadB coding region. The N696A primer included an XbaI site for cloning. The 1.1 kb PCR product was digested with AvrII and XbaI (New England Biolabs, Beverly, Mass.) and gel purified using a Qiaquick Gel Extraction Kit (Qiagen Inc., Valencia, Calif.)). The purified fragment was ligated with pTrc99A::budB-ilvC-ilvD-kivD, that had been cut with the same restriction enzymes, using T4 DNA ligase (New England Biolabs, Beverly, Mass.). The ligation mixture was incubated at 16° C. overnight and then transformed into E. coli Mach 1™ competent cells (Invitrogen) according to the manufacturer's protocol. Transformants were obtained following growth on the LB agar with 100 μg/ml ampicillin. Plasmid DNA from the transformants was prepared with QIAprep Spin Miniprep Kit (Qiagen Inc., Valencia, Calif.) according to manufacturer's protocols. The resulting plasmid was called pTrc99A::budB-ilvC-ilvD-kivD-sadB. Electrocompetent 4KO cells were prepared as described and transformed with pTrc99A::budB-ilvC-ilvD-kivD-sadB. Transformants were streaked onto LB agar plates containing 100 μg/mL ampicillin. The resulting strain carrying plasmid pTrc99A::budB-ilvC-ilvD-kivD-sadB with 4KO (designated strain NGCI-031) was used for fermentation studies outlined in Example 3.

### Example 3

### Production of Isobutanol by Recombinant E. coli Using Extractive Fermentation

The purpose of this Example is to demonstrate production of isobutanol by E. coli strain NGCI-031, constructed as described herein above. All seed cultures for inoculum preparation were grown in the LB medium with ampicillin (100 mg/L) as the selection antibiotic. The composition of the semi-synthetic medium used for this fermentation and the formulation of the trace metals used are given in Tables 3 and 4 below.

TABLE 3
Fermentation Medium Composition

| Ingredient | Amount/L | |
|---|---|---|
| 1 - Phosphoric Acid 85% | 0.75 | mL |
| 2 - Sulfuric Acid (18 M) | 0.30 | mL |
| 3 - Balch's w/ Cobalt - 1000X (see Table 4) | 1.00 | mL |
| 4 - Potassium Phosphate Monobasic | 1.40 | g |
| 5 - Citric Acid Monohydrate | 200 | g |
| 6 - Magnesium Sulfate, heptahydrate | 200 | g |
| 7 - Ferric Ammonium Citrate | 0.33 | g |
| 8 - Calcium chloride, dihydrate | 0.20 | g |
| 9 - Yeast Extract^{a} | 5.00 | g |
| 10 - Antifoam 204^{b} | 0.20 | mL |
| 11 - Thiamince•HCl, 5 g/L stock | 1.00 | mL |
| 12 - Ampicillin, 25 mg/mL stock | 4.00 | mL |
| 13 - Glucose 50 wt % stock | 33.3 | mL |

^{a}Obtained from BD Diagnostic Systems, Sparks, MD
^{b}the technical grade oleyl alcohol, which contained (65%) and higher and lower fatty alcohols, was obtained from Sigma-Aldrich (St Louis, MO) and used without further purification.

TABLE 4
Balch's modified trace metals - 1000X

| Ingredient | Concentration (g/L) |
|---|---|
| Citric Acid Monohydrate | 40.0 |
| MnSO₄•H₂O | 30.0 |
| NaCl | 10.0 |
| FeSO₄•7H₂O | 1.0 |
| CoCl₂•6H₂O | 1.0 |
| ZnSO₄•7H₂O | 1.5 |
| CuSO₄•5H₂O | 0.1 |
| Boric Acid (H₃BO₃) | 0.1 |
| Sodium Molybnate (NaMoO₄•2H₂O) | 0.1 |

Ingredients 1-10 from Table 3 were added to water at the prescribed concentration to make a final volume of 1.5 L in the fermentor and the contents of the fermentor were sterilized by autoclaving. Components 11-13 were mixed, filter sterilized and then added to the fermentor after the autoclaved medium had been cooled. The total final volume of the fermentation medium (the aqueous phase) was about 1.6 L.

A 3-L Biostat-B DCU-3 fermentor (Braun Biotech International, Melesungen, Germany) with a working volume of 2.0 L was used for fermentation while maintaining the temperature at 30° C. and the pH at 6.8 using ammonium hydroxide. Following inoculation of the medium with seed culture (2-10 vol %), the fermentor was operated aerobically at a 30% dissolved oxygen (DO) set point with 0.5 vvm of air flow while the agitation rate (rpm) was controlled automatically. The culture was induced with 0.4-0.5 mM IPTG to overexpress the isobutanol pathway once it reached to OD₆₀₀ of 10. Fermentation conditions were switched to microaerobic by decreasing the stirrer speed to 200 rpm 4 hr post induction. The shift to microaerobic conditions initiated isobutanol production while minimizing the incorporation of carbon to produce biomass, thereby uncoupling biomass formation from isobutanol production. Oleyl alcohol (about 780 mL) was added during the isobutanol production phase to alleviate product-induced inhibition due to build up of isobutanol in the aqueous phase. Glucose was added as a bolus (50 wt % stock solution) to the fermentor to keep glucose levels between 30 g/L and 2 g/L.

Since efficient production of isobutanol requires microaerobic conditions to enable redox balance in the biosynthetic pathway, air was continuously supplied to the fermentor at 0.5 vvm. Continuous aeration led to significant stripping of isobutanol from the aqueous phase of the fermentor. To determine the loss of isobutanol due to stripping, the off-gas from the fermentor was sparged through a chilled (6.5° C.) water trap to condense the isobutanol, which was then quantified using mass spectrometry using a Prima dB mass spectrometer (Thermo Electron Corp., Madison, Wis.). The isobutanol peaks at mass to charge ratios of 74 or 42 were used to determine the amount of isobutanol present.

Glucose and organic acids in the aqueous phase were routinely monitored during fermentation using a BioProfile® 300 Analyzer (Nova Biomedical, Waltham, Mass.). Glucose was also monitored using a glucose analyzer (YSI, Inc., Yellow Springs, Ohio). Isobutanol in the aqueous phase and isobutanol in the oleyl alcohol phase were monitored using gas chromatography (GC) as described below. The two phases were separated by centrifugation. The GC analysis was performed as described above. The effective titer, rate, and yield for isobutanol production, which were corrected for the isobutanol lost due to stripping, were 35 g/L, 0.40 g/L/h, and 0.33 g/g, respectively. The use of oleyl alcohol in an extractive fermentation for isobutanol production, due to extraction of the toxic isobutanol product from the fermentation medium and the host strain, results in significantly higher effective titer, rate, and yield.

### Example 4

The purpose of this example is to compare the effects on isobutanol production, of deletions in genes encoding pyruvate formate lyase, fumarate reductase, alcohol dehydrogenase and lactate dehydrogenase in an E. coli host vs. a host that does not have these deletions.

In order to compare the production of isobutanol by E. coli strain NGCI-031 (comprising deletions in pfIB, frdB, IdhA, and adhE genes) with that of an E. coli strain without deletions to pfIB, frdB, IdhA, and adhE genes, E. coli strain MG1655 (ATCC 47076) was transformed with plasmid pTrc99A::budB-ilvC-ilvD-kivD-sadB to produce E. coli strain MG1655/pTrc99A::budB-ilvC-ilvD-kivD-sadB. Fermentations were performed essentially as described above but without oleyl alcohol. The effective titer, rate, and yield for isobutanol production for strain NGCI-031 (which were corrected for the isobutanol lost due to stripping) were 11 g/L, 0.23 g/L/h, and 0.25 g/g, respectively; whereas, the effective titer, rate, and yield for isobutanol production for strain MG1655/pTrc99A::budB-ilvC-ilvD-kivD-sadB (which were corrected for the isobutanol lost due to stripping) were 14 g/L, 0.18 g/L/h, and 0.12 g/g, respectively. Deletions in pfIB, frdB, IdhA, and adhE led to significantly improved rate and yield compared to the strain without deletions in pfIB, frdB, IdhA, and adhE; the lower titer for the pfIB, frdB, IdhA, and adhE deleted strain was a result of shorter fermentation time.

What is claimed is:

1. An enteric production host for the production of isobutanol comprising at least one gene encoding a polypeptide having butanol dehydrogenase activity wherein the host produces isobutanol and is substantially free of at least one of the following enzyme activities:
a) Pyruvate formate lyase (EC 2.3.1.54)
b) Fumarate reductase (EC 1.3.99.1),
c) Alcohol dehydrogenase (EC 1.2.1.10/EC 1.1.1.1)
d) Lactate dehydrogenase (EC 1.1.1.28)

2. The enteric production host of claim 1 wherein the at least one gene encoding a polypeptide having butanol dehydrogenase activity is isolated from is isolated from A. xylosoxidans.

3. The enteric production host of claim 2 wherein the at least one gene encoding a polypeptide having butanol dehydrogenase encodes a polypeptide having at least 90% identity to the amino acid sequence as set forth in SEQ ID NO: 10 over a length of 348 amino acids using BLAST with scoring matrix BLOSUM62, an expect cutoff of 10 and word size 3 and a gap opening penalty of 11 and a gap extension of 1.

4. The enteric production host of claim 1 wherein the host cell is a member of a genus selected from the group consisting of Escherichia, Salmonella, Erwinia, Shigella, Kelbsiella, Serratia.

5. The enteric production host of claim 4 wherein the host is an E. coli.

6. The enteric production host of claim 5 comprising a deletion in at least one endogenous gene encoding an enzyme or a portion of an enzyme selected from the group consisting of: Pyruvate formate lyase (EC 2.3.1.54), Fumarate reductase (EC 1.3.99.1), Alcohol dehydrogenase (EC 1.2.1.10/EC 1.1.1.1), and Lactate dehydrogenase (EC 1.1.1.28).

7. The enteric production host of claim 6 wherein the pyruvate formate lyase has the amino acid sequence as set forth in SEQ ID NO:46.

8. The enteric production host of claim 6 wherein the fumarate reductase has the amino acid selected from the group consisting of SEQ ID NO: 54, 48, 56, and 58.

9. The enteric production host of claim 6 wherein the alcohol dehydrogenase has the amino acid sequence as set forth in SEQ ID NO: 52.

10. The enteric production host of claim 6 wherein the lactate dehydrogenase has the amino acid sequence as set forth in SEQ ID NO: 50.

11. The enteric production host of claims 1 or 6 having an isobutanol biosynthetic pathway comprising:
a) at least one gene encoding an acetolactate synthase having the EC number 2.2.1.6 9 for conversion of pyruvate to acetolactate:
b) at least one gene encoding acetohydroxy acid isomeroreductase having the EC number 1.1.1.86 for conversion of acetolactate to 2,3-dihydroxyisovalerate;
c) at least one gene encoding acetohydroxy acid dehydratase having the EC number 4.2.1.9 for conversion of 2,3-dihydroxyisovalerate to α-ketoisovalerate;
d) at least one gene encoding a branched-chain keto acid decarboxylase having the EC number 4.1.1.72 for conversion of α-ketoisovalerate to isobutyraldehyde; and
e) at least one gene encoding a butanol dehydrogenase polypeptide that functions to catalyze the reaction of isobutyraldehyde to isobutanol, wherein the butanol dehydrogenase polypeptide is a butanol dehydrogenase of claim 3.

12. The enteric production host of claim 11 wherein the acetolactate synthase has an amino acid sequence selected from the group consisting of SEQ ID NO:11, SEQ ID NO:2, and SEQ ID NO:13.

13. The enteric production host of claim 11 wherein the acetohydroxy acid isomeroreductase has an amino acid sequence selected from the group consisting of SEQ ID NO: 15, 17 and 19

14. The enteric production host of claim 11 wherein the acetohydroxy acid dehydratase has an amino acid sequence selected from the group consisting of SEQ ID NO: 6, 21, 23, and 25.

15. The enteric production host of claim 11 wherein the branched-chain keto acid decarboxylase has an amino acid sequence selected from the group consisting of SEQ ID NOs: 27, 8, 30, and 32.

16. A method for the production of isobutanol comprising growing the production host of claims 1 or 6 in a fermentation medium comprising a carbon substrate under conditions wherein isobutanol is produced.


SEQUENCE LISTING (59 sequences)

SEQ ID NO: 1 - DNA, 1680 bp, Klebsiella pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 2 - protein, 559 aa, klebsiella pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 3 - DNA, 1476 bp, Escherichia coli
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 4 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 5 - DNA, 1852 bp, Escherichia coli
aatgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 6 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 7 - DNA, 1662 bp, artificial sequence
  codon optimized kivD gene of Lactococcuslactis
tctagacatatgtatactgtgggggattacctgctggatcgcctgcacgaactggggattgaagaaattttcggtgtgccaggcgattataacctgcagttcctggaccagattatctcgcacaaagatatgaagtgggtcggtaacgccaacgaactgaacgcgagctatatggcagatggttatgcccgtaccaaaaaagctgctgcgtttctgacgacctttggcgttggcgaactgagcgccgtcaacggactggcaggaagctacgccgagaacctgccagttgtcgaaattgttgggtcgcctacttctaaggttcagaatgaaggcaaatttgtgcaccatactctggctgatggggattttaaacattttatgaaaatgcatgaaccggttactgcggcccgcacgctgctgacagcagagaatgctacggttgagatcgaccgcgtcctgtctgcgctgctgaaagagcgcaagccggtatatatcaatctgcctgtcgatgttgccgcagcgaaagccgaaaagccgtcgctgccactgaaaaaagaaaacagcacctccaatacatcggaccaggaaattctgaataaaatccaggaatcactgaagaatgcgaagaaaccgatcgtcatcaccggacatgagatcatctcttttggcctggaaaaaacggtcacgcagttcatttctaagaccaaactgcctatcaccaccctgaacttcggcaaatctagcgtcgatgaagcgctgccgagttttctgggtatctataatggtaccctgtccgaaccgaacctgaaagaattcgtcgaaagcgcggactttatcctgatgctgggcgtgaaactgacggatagctccacaggcgcatttacccaccatctgaacgagaataaaatgatttccctgaatatcgacgaaggcaaaatctttaacgagcgcatccagaacttcgattttgaatctctgattagttcgctgctggatctgtccgaaattgagtataaaggtaaatatattgataaaaaacaggaggattttgtgccgtctaatgcgctgctgagtcaggatcgtctgtggcaagccgtagaaaacctgacacagtctaatgaaacgattgttgcggaacagggaacttcatttttcggcgcctcatccatttttctgaaatccaaaagccatttcattggccaaccgctgtgggggagtattggttatacctttccggcggcgctgggttcacagattgcagataaggaatcacgccatctgctgtttattggtgacggcagcctgcagctgactgtccaggaactggggctggcgatccgtgaaaaaatcaatccgatttgctttatcatcaataacgacggctacaccgtcgaacgcgaaattcatggaccgaatcaaagttacaatgacatcccgatgtggaactatagcaaactgccggaatcctttggcgcgacagaggatcgcgtggtgagtaaaattgtgcgtacggaaaacgaatttgtgtcggttatgaaagaagcgcaggctgacccgaatcgcatgtattggattgaactgatcctggcaaaagaaggcgcaccgaaagttctgaaaaagatggggaaactgtttgcggagcaaaataaaagctaaggatcc

SEQ ID NO: 8 - protein, 548 aa, artificial sequence
  amino acid sequence of the codon optimized kivD gene of lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 9 - DNA, 1047 bp, Achromobacter xylosoxidans
atgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctga

SEQ ID NO: 10 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 11 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 12 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 13 - protein, 554 aa, lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 14 - DNA, 1665 bp, lactococcus lactis
atgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattga

SEQ ID NO: 15 - protein, 395 aa, Saccharomyces cerevisiae
MLRTQAARLICNSRVITAKRTFALATRAAAYSRPAARFVKPMITTRGLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 16 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 17 - protein, 330 aa, Methanococcus maripaludis
MKVFYDSDFKLDALKEKTIAVIGYGSQGRAQSLNMKDSGLNVVVGLRKNGASWNNAKADGHNVMTIEEAAEKADIIHILIPDELQAEVYESQIKPYLKEGKTLSFSHGFNIHYGFIVPPKGVNVVLVAPKSPGKMVRRTYEEGFGVPGLICIEIDATNNAFDIVSAMAKGIGLSRAGVIQTTFKEETETDLFGEQAVLCGGVTELIKAGFETLVEAGYAPEMAYFETCHELKLIVDLIYQKGFKNMWNDVSNTAEYGGLTRRSRIVTADSKAAMKEILREIQDGRFTKEFLLEKQVSYAHLKSMRRLEGDLQIEEVGAKLRKMCGLEKEE

SEQ ID NO: 18 - DNA, 993 bp, Methanococcus maripaludis
atgaaggtattctatgactcagattttaaattagatgctttaaaagaaaaaacaattgcagtaatcggttatggaagtcaaggtagggcacagtccttaaacatgaaagacagcggattaaacgttgttgttggtttaagaaaaaacggtgcttcatggaacaacgctaaagcagacggtcacaatgtaatgaccattgaagaagctgctgaaaaagcggacatcatccacatcttaatacctgatgaattacaggcagaagtttatgaaagccagataaaaccatacctaaaagaaggaaaaacactaagcttttcacatggttttaacatccactatggattcattgttccaccaaaaggagttaacgtggttttagttgctccaaaatcacctggaaaaatggttagaagaacatacgaagaaggtttcggtgttccaggtttaatctgtattgaaattgatgcaacaaacaacgcatttgatattgtttcagcaatggcaaaaggaatcggtttatcaagagctggagttatccagacaactttcaaagaagaaacagaaactgaccttttcggtgaacaagctgttttatgcggtggagttaccgaattaatcaaggcaggatttgaaacactcgttgaagcaggatacgcaccagaaatggcatactttgaaacctgccacgaattgaaattaatcgttgacttaatctaccaaaaaggattcaaaaacatgtggaacgatgtaagtaacactgcagaatacggcggacttacaagaagaagcagaatcgttacagctgattcaaaagctgcaatgaaagaaatcttaagagaaatccaagatggaagattcacaaaagaattccttctcgaaaaacaggtaagctatgctcatttaaaatcaatgagaagactcgaaggagacttacaaatcgaagaagtcggcgcaaaattaagaaaaatgtgcggtcttgaaaaagaagaataa

SEQ ID NO: 19 - protein, 342 aa, Bacillus subtilis
MVKVYYNGDIKENVLAGKTVAVIGYGSQGHAHALNLKESGVDVIVGVRQGKSFTQAQEDGHKVFSVKEAAAQAEIIMVLLPDEQQQKVYEAEIKDELTAGKSLVFAHGFNVHFHQIVPPADVDVFLVAPKGPGHLVRRTYEQGAGVPALFAIYQDVTGEARDKALAYAKGIGGARAGVLETTFKEETETDLFGEQAVLCGGLSALVKAGFETLTEAGYQPELAYFECLHELKLIVDLMYEEGLAGMRYSISDTAQWGDFVSGPRVVDAKVKESMKEVLKDIQNGTFAKEWIVENQVNRPRFNAINASENEHQIEVVGRKLREMMPFVKQGKKKEAVVSVAQN

SEQ ID NO: 20 - DNA, 1476 bp, Bacillus subtilis; identical to SEQ ID NO: 3

SEQ ID NO: 21 - protein, 585 aa, saccharomyces serevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 22 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 23 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 24 - DNA, 1653 bp, Methanococcus maripaludis
atgataagtgataacgtcaaaaagggagttataagaactccaaaccgagctcttttaaaggcttgcggatatacagacgaagacatggaaaaaccatttattggaattgtaaacagctttacagaagttgttcccggccacattcacttaagaacattatcagaagcggctaaacatggtgtttatgcaaacggtggaacaccatttgaatttaataccattggaatttgcgacggtattgcaatgggccacgaaggtatgaaatactctttaccttcaagagaaattattgcagacgctgttgaatcaatggcaagagcacatggatttgatggtcttgttttaattcctacgtgtgataaaatcgttcctggaatgataatgggtgctttaagactaaacattccatttattgtagttactggaggaccaatgcttcccggagaattccaaggtaaaaaatacgaacttatcagcctttttgaaggtgtcggagaataccaagttggaaaaattactgaagaagagttaaagtgcattgaagactgtgcatgttcaggtgctggaagttgtgcagggctttacactgcaaacagtatggcctgccttacagaagctttgggactctctcttccaatgtgtgcaacaacgcatgcagttgatgcccaaaaagttaggcttgctaaaaaaagtggctcaaaaattgttgatatggtaaaagaagacctaaaaccaacagacatattaacaaaagaagcttttgaaaatgctattttagttgaccttgcacttggtggatcaacaaacacaacattacacattcctgcaattgcaaatgaaattgaaaataaattcataactctcgatgactttgacaggttaagcgatgaagttccacacattgcatcaatcaaaccaggtggagaacactacatgattgatttacacaatgctggaggtattcctgcggtattgaacgttttaaaagaaaaaattagagatacaaaaacagttgatggaagaagcattttggaaatcgcagaatctgttaaatacataaattacgacgttataagaaaagtggaagctccggttcacgaaactgctggtttaagggttttaaagggaaatcttgctccaaacggttgcgttgtaaaaatcggtgcagtacatccgaaaatgtacaaacacgatggacctgcaaaagtttacaattccgaagatgaagcaatttctgcgatacttggcggaaaaattgtagaaggggacgttatagtaatcagatacgaaggaccatcaggaggccctggaatgagagaaatgctctccccaacttcagcaatctgtggaatgggtcttgatgacagcgttgcattgattactgatggaagattcagtggtggaagtaggggcccatgtatcggacacgtttctccagaagctgcagctggcggagtaattgctgcaattgaaaacggggatatcatcaaaatcgacatgattgaaaaagaaataaatgttgatttagatgaatcagtcattaaagaaagactctcaaaactgggagaatttgagcctaaaatcaaaaaaggctatttatcaagatactcaaaacttgtctcatctgctgacgaaggggcagttttaaaataa

SEQ ID NO: 25 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSYGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 26 - DNA, 1677 bp, Bacillus subtilis
atggcagaattacgcagtaatatgatcacacaaggaatcgatagagctccgcaccgcagtttgcttcgtgcagcaggggtaaaagaagaggatttcggcaagccgtttattgcggtgtgtaattcatacattgatatcgttcccggtcatgttcacttgcaggagtttgggaaaatcgtaaaagaagcaatcagagaagcagggggcgttccgtttgaatttaataccattggggtagatgatggcatcgcaatggggcatatcggtatgagatattcgctgccaagccgtgaaattatcgcagactctgtggaaacggttgtatccgcacactggtttgacggaatggtctgtattccgaactgcgacaaaatcacaccgggaatgcttatggcggcaatgcgcatcaacattccgacgatttttgtcagcggcggaccgatggcggcaggaagaacaagttacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtgtttattgaagatcttcacgaagcgggcggcgtttcagcggctctgaatgagctttcgaagaaagaaggagcgcttcatttagatgcgctgactgttacaggaaaaactcttggagaaaccattgccggacatgaagtaaaggattatgacgtcattcacccgctggatcaaccattcactgaaaagggaggccttgctgttttattcggtaatctagctccggacggcgctatcattaaaacaggcggcgtacagaatgggattacaagacacgaagggccggctgtcgtattcgattctcaggacgaggcgcttgacggcattatcaaccgaaaagtaaaagaaggcgacgttgtcatcatcagatacgaagggccaaaaggcggacctggcatgccggaaatgctggcgccaacatcccaaatcgttggaatgggactcgggccaaaagtggcattgattacggacggacgtttttccggagcctcccgtggcctctcaatcggccacgtatcacctgaggccgctgagggcgggccgcttgcctttgttgaaaacggagaccatattatcgttgatattgaaaaacgcatcttggatgtacaagtgccagaagaagagtgggaaaaacgaaaagcgaactggaaaggttttgaaccgaaagtgaaaaccggctacctggcacgttattctaaacttgtgacaagtgccaacaccggcggtattatgaaaatctag

SEQ ID NO: 27 - protein, 547 aa, lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 28 - DNA, 1644 bp, lactococcus lactis
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 29 - DNA, 1647 bp, lactococcus lactis
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 30 - protein, 550 aa, salmonella typhimurium
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 31 - DNA, 1653 bp, salmonella typhimurium
ttatcccccgttgcgggcttccagcgcccgggtcacggtacgcagtaattccggcagatcggcttttggcaacatcacttcaataaatgacagacgttgtgggcgcgccaaccgttcgaggacctctgccagttggatagcctgcgtcacccgccagcactccgcctgttgcgccgcgtttagcgccggtggtatctgcgtccagttccagctcgcgatgtcgttataccgctgggccgcgccgtgaatggcgcgctctacggtatagccgtcattgttgagcagcaggatgaccggcgcctgcccgtcgcgtaacatcgagcccatctcctgaatcgtgagctgcgccgcgccatcgccgataatcagaatcacccgccgatcgggacaggcggtttgcgcgccaaacgcggcgggcaaggaatagccgatagacccccacagcggctgtaacacaacttccgcgccgtcaggaagcgacagcgcggcagcgccaaaagctgctgtcccctggtcgacaaggataatatctccgggtttgagatactgctgtaaggtttgccagaagctttcctgggtcagttctcctttatcaatccgcactggctgtccggcggaacgcgtcggcggcggcgcaaaagcgcattccaggcacagttcgcgcagcgtagacaccgcctgcgccatcgggaggttgaaccaggtttcgccgatgcgcgacgcgtaaggctgaatctccagcgtgcgttccgccggtaattgttgggtaaatccggccgtaagggtatcgacaaaacgggtgccgacgcagataaccctatcggcgtcctctatggcctgacgcacttctttgctgctggcgccagcgctataggtgccaacgaagttcgggtgctgttcatcaaaaagccccttccccatcagtagtgtcgcatgagcgatgggcgtttccgccatccagcgctgcaacagtggtcgtaaaccaaaacgcccggcaagaaagtcggccaatagcgcaatgcgccgactgttcatcaggcactgacgggcgtgataacgaaaggccgtctccacgccgctttgcgcttcatgcacgggcaacgccagcgcctgcgtaggtgggatggccgtttttttcgccacatcggcgggcaacatgatgtatcctggcctgcgtgcggcaagcatttcacccaacacgcggtcaatctcgaaacaggcgttctgttcatctaatattgcgctggcagcggatatcgcctgactcatgcgataaaaatgacgaaaatcgccgtcaccgagggtatggtgcatcaattcgccacgctgctgcgcagcgctacagggcgcgccgacgatatgcaagaccgggacatattccgcgtaactgcccgcgataccgttaatagcgctaagttctcccacgccaaaggtggtgagtagcgctccagcgcccgacatgcgcgcatagccgtccgcggcataagcggcgttcagctcattggcgcatcccacccaacgcagggtcgggtggtcaatcacatggtcaagaaactgcaagttataatcgcccggtacgccaaaaagatggccaatgccgcatcctgccagtctgtccagcaaatagtcggccacggtataggggttttgcat

SEQ ID NO: 32 - protein, 554 aa, clostridium acetobutylicum
MKSEYTIGRYLLDRLSELGIRHIFGVPGDYNLSFLDYIMEYKGIDWVGNCNELNAGYAADGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNN

SEQ ID NO: 33 - DNA, 1665 bp, clostridium acetobutylicum
ttgaagagtgaatacacaattggaagatatttgttagaccgtttatcagagttgggtattcggcatatctttggtgtacctggagattacaatctatcctttttagactatataatggagtacaaagggatagattgggttggaaattgcaatgaattgaatgctgggtatgctgctgatggatatgcaagaataaatggaattggagccatacttacaacatttggtgttggagaattaagtgccattaacgcaattgctggggcatacgctgagcaagttccagttgttaaaattacaggtatccccacagcaaaagttagggacaatggattatatgtacaccacacattaggtgacggaaggtttgatcacttttttgaaatgtttagagaagtaacagttgctgaggcattactaagcgaagaaaatgcagcacaagaaattgatcgtgttcttatttcatgctggagacaaaaacgtcctgttcttataaatttaccgattgatgtatatgataaaccaattaacaaaccattaaagccattactcgattatactatttcaagtaacaaagaggctgcatgtgaatttgttacagaaatagtacctataataaatagggcaaaaaagcctgttattcttgcagattatggagtatatcgttaccaagttcaacatgtgcttaaaaacttggccgaaaaaaccggatttcctgtggctacactaagtatgggaaaaggtgttttcaatgaagcacaccctcaatttattggtgtttataatggtgatgtaagttctccttatttaaggcagcgagttgatgaagcagactgcattattagcgttggtgtaaaattgacggattcaaccacagggggattttctcatggattttctaaaaggaatgtaattcacattgatcctttttcaataaaggcaaaaggtaaaaaatatgcacctattacgatgaaagatgctttaacagaattaacaagtaaaattgagcatagaaactttgaggatttagatataaagccttacaaatcagataatcaaaagtattttgcaaaagagaagccaattacacaaaaacgtttttttgagcgtattgctcactttataaaagaaaaagatgtattattagcagaacagggtacatgcttttttggtgcgtcaaccatacaactacccaaagatgcaacttttattggtcaacctttatggggatctattggatacacacttcctgctttattaggttcacaattagctgatcaaaaaaggcgtaatattcttttaattggggatggtgcatttcaaatgacagcacaagaaatttcaacaatgcttcgtttacaaatcaaacctattatttttttaattaataacgatggttatacaattgaacgtgctattcatggtagagaacaagtatataacaatattcaaatgtggcgatatcataatgttccaaaggttttaggtcctaaagaatgcagcttaacctttaaagtacaaagtgaaactgaacttgaaaaggctcttttagtggcagataaggattgtgaacatttgatttttatagaagttgttatggatcgttatgataaacccgagcctttagaacgtctttcgaaacgttttgcaaatcaaaataattag

SEQ ID NO: 34 - DNA, 24 bp, artificial seqeunce
  primer sequence
tcatcactgataacctgattccgg

SEQ ID NO: 35 - DNA, 26 bp, artificial sequence
  primer
cgagtctgttttggcagtcaccttaa

SEQ ID NO: 36 - DNA, 23 bp, artificial sequence
  primer
gagcgtgacgacgtcaacttcct

SEQ ID NO: 37 - DNA, 23 bp, artificial sequence
  primer
cagttcaatgctgaaccacacag

SEQ ID NO: 38 - DNA, 23 bp, artificial sequence
  primer
gaaggttgcgcctacactaagca

SEQ ID NO: 39 - DNA, 23 bp, artificial sequence
  primer
gggagcggcaagattaaaccagt

SEQ ID NO: 40 - DNA, 23 bp, artificial sequence
  primer
tggatcacgtaatcagtacccag

SEQ ID NO: 41 - DNA, 23 bp, artificial sequence
  primer
atccttaactgatcggcattgcc

SEQ ID NO: 42 - DNA, 30 bp, artificial sequence
  primer
gacctaggaggtcacacatgaaagctctgg

SEQ ID NO: 43 - DNA, 25 bp, artificial sequence
  primer
cgactctagaggatccccgggtacc

SEQ ID NO: 44 - DNA, 30 bp, artificial sequence
  primer
ggaattcacacatgaaagctctggtttatc

SEQ ID NO: 45 - DNA, 28 bp, artificial sequence
  primer
gcgtccagggcgtcaaagatcaggcagc

SEQ ID NO: 46 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 47 - DNA, 2283 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 48 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 49 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 50 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 51 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 52 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 53 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 54 - protein, 602 aa, Escherichia coli
MQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 55 - DNA, 1809 bp, Escherichia coli
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 56 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 57 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 58 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 59 - DNA, 360 bp, Escherichia coli
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa